(19) United States
(12) Patent Application Publication
(10) Pub. No.: US 2026/0285922 A1
(43) Pub. Date: Sep. 24, 2026

(54) Optogenetic Actuators and Related Nucleic Acids, Constructs, Compositions, and Methods

(71) Applicant: Science Corporation, Alameda, CA (US)

(72) Inventors: Alexander Naka, San Francisco, CA (US); Alan Mardinly, Oakland, CA (US); Amanda Tose, Alameda, CA (US); Alberto Nava, Berkeley, CA (US)

(21) Appl. No.: 19/570,871

(22) Filed: Mar. 18, 2026

Related U.S. Application Data
(60) Provisional application No. 63/773,874, filed on Mar. 18, 2025.
(60) Provisional application No. 63/879,994, filed on Sep. 11, 2025.

(51) Int. Cl. C07K 14/44; A61K 35/76; A61K 38/00; A61K 48/00; C12N 15/86

(52) U.S. Cl. CPC C07K 14/44; A61K 35/76; A61K 48/0008; A61K 48/005; C12N 15/86; A61K 38/00; C12N 2750/14143

(57) ABSTRACT

The disclosure relates to engineered WiChR having desirable functional properties that make them suitable for optogenetic applications, including use in restoration of vision applications. Nucleic acids that encode WiChR variants, genetic constructs that include a nucleic acid that encodes a WiChR variant, compositions for expressing WiChR variants, and methods of treatment are also described.

### FIELD

The disclosure relates generally to the field of optogenetics. More particularly, the disclosure relates to channelrhodopsins useful as optogenetic actuators. Specific examples relate to nucleic acids that encode a channelrhodopsin, an amino acid sequence for a channelrhodopsin, fusion proteins, genetic constructs that include a nucleic acid encoding a channelrhodopsin, compositions that include a nucleic acid encoding a channelrhodopsin, and methods of treatment.

### SEQUENCE LISTING

The disclosure includes a Sequence Listing which has been submitted as a Sequence Listing XML via Patent Center and is hereby incorporated by reference in its entirety. The Sequence Listing XML file is named 70811-US-NP-sequence-listing.xml, was created on Mar. 23, 2026, and is 510,101 bytes in size.

### BACKGROUND

Optogenetics has emerged as a powerful tool for controlling cellular processes with high spatiotemporal precision using light-sensitive proteins. Among these, channelrhodopsins are key optogenetic actuators that enable the modulation of cellular activity through light stimulation.

Despite recent advances, there remains a need for improved optogenetic actuators that exhibit enhanced performance characteristics, including greater expression efficiency, stability, and functional selectivity.

### BRIEF SUMMARY OF SELECTED EXAMPLES

The disclosure provides novel engineered channelrhodopsins. Certain embodiments described herein are derived from WiChr, a natural channelrhodopsin from Wobblia lunata, a microscopic marine flagellate. Various embodiments include alterations of a natural sequence that alters one or more properties of a natural channelrhodopsin, such as WiChr. Additional embodiments relate to nucleic acids that encode a WiChR variant, genetic constructs that include a nucleic acid encoding a WiChR variant, fusion proteins that include a WiChR variant, compositions that include a nucleic acid encoding a WiChR variant, and methods of treatment.

Various example nucleic acids that encode a WiChR variant are described herein.

An example nucleic acid that encodes a WiChR variant comprises the nucleic acid set forth in SEQ ID NO: 3. Another example nucleic acid that encodes a WiChR variant comprises the nucleic acid set forth in SEQ ID NO: 5. Another example nucleic acid that encodes a WiChR variant comprises the nucleic acid set forth in SEQ ID NO: 7. Another example nucleic acid that encodes a WiChR variant comprises the nucleic acid set forth in SEQ ID NO: 9. Another example nucleic acid that encodes a WiChR variant comprises the nucleic acid set forth in SEQ ID NO: 11. Another example nucleic acid that encodes a WiChR variant comprises the nucleic acid set forth in SEQ ID NO: 13. Another example nucleic acid that encodes a WiChR variant comprises the nucleic acid set forth in SEQ ID NO: 15. Another example nucleic acid that encodes a WiChR variant comprises the nucleic acid set forth in SEQ ID NO: 17. Another example nucleic acid that encodes a WiChR variant comprises the nucleic acid set forth in SEQ ID NO: 19. Another example nucleic acid that encodes a WiChR variant comprises the nucleic acid set forth in SEQ ID NO: 21. Another example nucleic acid that encodes a WiChR variant comprises the nucleic acid set forth in SEQ ID NO: 23. Another example nucleic acid that encodes a WiChR variant comprises the nucleic acid set forth in SEQ ID NO: 25. Another example nucleic acid that encodes a WiChR variant comprises the nucleic acid set forth in SEQ ID NO: 27. Another example nucleic acid that encodes a WiChR variant comprises the nucleic acid set forth in SEQ ID NO: 29. Another example nucleic acid that encodes a WiChR variant comprises the nucleic acid set forth in SEQ ID NO: 31. Another example nucleic acid that encodes a WiChR variant comprises the nucleic acid set forth in SEQ ID NO: 33. Another example nucleic acid that encodes a WiChR variant comprises the nucleic acid set forth in SEQ ID NO: 35. Another example nucleic acid that encodes a WiChR variant comprises the nucleic acid set forth in SEQ ID NO: 37. Another example nucleic acid that encodes a WiChR variant comprises the nucleic acid set forth in SEQ ID NO: 39. Another example nucleic acid that encodes a WiChR variant comprises the nucleic acid set forth in SEQ ID NO: 41. Another example nucleic acid that encodes a WiChR variant comprises the nucleic acid set forth in SEQ ID NO: 43. Another example nucleic acid that encodes a WiChR variant comprises the nucleic acid set forth in SEQ ID NO: 45. Another example nucleic acid that encodes a WiChR variant comprises the nucleic acid set forth in SEQ ID NO: 47. Another example nucleic acid that encodes a WiChR variant comprises the nucleic acid set forth in SEQ ID NO: 49. Another example nucleic acid that encodes a WiChR variant comprises the nucleic acid set forth in SEQ ID NO: 51. Another example nucleic acid that encodes a WiChR variant comprises the nucleic acid set forth in SEQ ID NO: 53. Another example nucleic acid that encodes a WiChR variant comprises the nucleic acid set forth in SEQ ID NO: 55. Another example nucleic acid that encodes a WiChR variant comprises the nucleic acid set forth in SEQ ID NO: 57. Another example nucleic acid that encodes a WiChR variant comprises the nucleic acid set forth in SEQ ID NO: 59. Another example nucleic acid that encodes a WiChR variant comprises the nucleic acid set forth in SEQ ID NO: 61. Another example nucleic acid that encodes a WiChR variant comprises the nucleic acid set forth in SEQ ID NO: 63. Another example nucleic acid that encodes a WiChR variant comprises the nucleic acid set forth in SEQ ID NO: 65. Another example nucleic acid that encodes a WiChR variant comprises the nucleic acid set forth in SEQ ID NO: 67. Another example nucleic acid that encodes a WiChR variant comprises the nucleic acid set forth in SEQ ID NO: 69. Another example nucleic acid that encodes a WiChR variant comprises the nucleic acid set forth in SEQ ID NO: 71. Another example nucleic acid that encodes a WiChR variant comprises the nucleic acid set forth in SEQ ID NO: 73. Another example nucleic acid that encodes a WiChR variant comprises the nucleic acid set forth in SEQ ID NO: 75. Another example nucleic acid that encodes a WiChR variant comprises the nucleic acid set forth in SEQ ID NO: 77. Another example nucleic acid that encodes a WiChR variant comprises the nucleic acid set forth in SEQ ID NO: 79. Another example nucleic acid that encodes a WiChR variant comprises the nucleic acid set forth in SEQ ID NO: 81. Another example nucleic acid that encodes a WiChR variant comprises the nucleic acid set forth in SEQ ID NO: 83. Another example nucleic acid that encodes a WiChR variant comprises the nucleic acid set forth in SEQ ID NO: 85. Another example nucleic acid that encodes a WiChR variant comprises the nucleic acid set forth in SEQ ID NO: 87. Another example nucleic acid that encodes a WiChR variant comprises the nucleic acid set forth in SEQ ID NO: 89. Another example nucleic acid that encodes a WiChR variant comprises the nucleic acid set forth in SEQ ID NO: 91. Another example nucleic acid that encodes a WiChR variant comprises the nucleic acid set forth in SEQ ID NO: 93. Another example nucleic acid that encodes a WiChR variant comprises the nucleic acid set forth in SEQ ID NO: 95. Another example nucleic acid that encodes a WiChR variant comprises the nucleic acid set forth in SEQ ID NO: 97. Another example nucleic acid that encodes a WiChR variant comprises the nucleic acid set forth in SEQ ID NO: 99. Another example nucleic acid that encodes a WiChR variant comprises the nucleic acid set forth in SEQ ID NO: 101. Another example nucleic acid that encodes a WiChR variant comprises the nucleic acid set forth in SEQ ID NO: 103. Another example nucleic acid that encodes a WiChR variant comprises the nucleic acid set forth in SEQ ID NO: 105. Another example nucleic acid that encodes a WiChR variant comprises the nucleic acid set forth in SEQ ID NO: 107. Another example nucleic acid that encodes a WiChR variant comprises the nucleic acid set forth in SEQ ID NO: 109. Another example nucleic acid that encodes a WiChR variant comprises the nucleic acid set forth in SEQ ID NO: 111. Another example nucleic acid that encodes a WiChR variant comprises the nucleic acid set forth in SEQ ID NO: 113. Another example nucleic acid that encodes a WiChR variant comprises the nucleic acid set forth in SEQ ID NO: 115. Another example nucleic acid that encodes a WiChR variant comprises the nucleic acid set forth in SEQ ID NO: 117. Another example nucleic acid that encodes a WiChR variant comprises the nucleic acid set forth in SEQ ID NO: 119. Another example nucleic acid that encodes a WiChR variant comprises the nucleic acid set forth in SEQ ID NO: 121. Another example nucleic acid that encodes a WiChR variant comprises the nucleic acid set forth in SEQ ID NO: 123. Another example nucleic acid that encodes a WiChR variant comprises the nucleic acid set forth in SEQ ID NO: 125. Another example nucleic acid that encodes a WiChR variant comprises the nucleic acid set forth in SEQ ID NO: 127. Another example nucleic acid that encodes a WiChR variant comprises the nucleic acid set forth in SEQ ID NO: 129.

Various example proteins that comprise a WiChR variant are described herein.

An example protein that comprises a WiChR variant comprises the amino acid sequence set forth in SEQ ID NO: 4. Another example protein that comprises a WiChR variant comprises the amino acid sequence set forth in SEQ ID NO: 6. Another example protein that comprises a WiChR variant comprises the amino acid sequence set forth in SEQ ID NO: 8. Another example protein that comprises a WiChR variant comprises the amino acid sequence set forth in SEQ ID NO: 10. Another example protein that comprises a WiChR variant comprises the amino acid sequence set forth in SEQ ID NO: 12. Another example protein that comprises a WiChR variant comprises the amino acid sequence set forth in SEQ ID NO: 14. Another example protein that comprises a WiChR variant comprises the amino acid sequence set forth in SEQ ID NO: 16. Another example protein that comprises a WiChR variant comprises the amino acid sequence set forth in SEQ ID NO: 18. Another example protein that comprises a WiChR variant comprises the amino acid sequence set forth in SEQ ID NO: 20. Another example protein that comprises a WiChR variant comprises the amino acid sequence set forth in SEQ ID NO: 22. Another example protein that comprises a WiChR variant comprises the amino acid sequence set forth in SEQ ID NO: 24. Another example protein that comprises a WiChR variant comprises the amino acid sequence set forth in SEQ ID NO: 26. Another example protein that comprises a WiChR variant comprises the amino acid sequence set forth in SEQ ID NO: 28. Another example protein that comprises a WiChR variant comprises the amino acid sequence set forth in SEQ ID NO: 30. Another example protein that comprises a WiChR variant comprises the amino acid sequence set forth in SEQ ID NO: 32. Another example protein that comprises a WiChR variant comprises the amino acid sequence set forth in SEQ ID NO: 34. Another example protein that comprises a WiChR variant comprises the amino acid sequence set forth in SEQ ID NO: 36. Another example protein that comprises a WiChR variant comprises the amino acid sequence set forth in SEQ ID NO: 38. Another example protein that comprises a WiChR variant comprises the amino acid sequence set forth in SEQ ID NO: 40. Another example protein that comprises a WiChR variant comprises the amino acid sequence set forth in SEQ ID NO: 42. Another example protein that comprises a WiChR variant comprises the amino acid sequence set forth in SEQ ID NO: 44. Another example protein that comprises a WiChR variant comprises the amino acid sequence set forth in SEQ ID NO: 46. Another example protein that comprises a WiChR variant comprises the amino acid sequence set forth in SEQ ID NO: 48. Another example protein that comprises a WiChR variant comprises the amino acid sequence set forth in SEQ ID NO: 50. Another example protein that comprises a WiChR variant comprises the amino acid sequence set forth in SEQ ID NO: 52. Another example protein that comprises a WiChR variant comprises the amino acid sequence set forth in SEQ ID NO: 54. Another example protein that comprises a WiChR variant comprises the amino acid sequence set forth in SEQ ID NO: 56. Another example protein that comprises a WiChR variant comprises the amino acid sequence set forth in SEQ ID NO: 58. Another example protein that comprises a WiChR variant comprises the amino acid sequence set forth in SEQ ID NO: 60. Another example protein that comprises a WiChR variant comprises the amino acid sequence set forth in SEQ ID NO: 62. Another example protein that comprises a WiChR variant comprises the amino acid sequence set forth in SEQ ID NO: 64. Another example protein that comprises a WiChR variant comprises the amino acid sequence set forth in SEQ ID NO: 66. Another example protein that comprises a WiChR variant comprises the amino acid sequence set forth in SEQ ID NO: 68. Another example protein that comprises a WiChR variant comprises the amino acid sequence set forth in SEQ ID NO: 70. Another example protein that comprises a WiChR variant comprises the amino acid sequence set forth in SEQ ID NO: 72. Another example protein that comprises a WiChR variant comprises the amino acid sequence set forth in SEQ ID NO: 74. Another example protein that comprises a WiChR variant comprises the amino acid sequence set forth in SEQ ID NO: 76. Another example protein that comprises a WiChR variant comprises the amino acid sequence set forth in SEQ ID NO: 78. Another example protein that comprises a WiChR variant comprises the amino acid sequence set forth in SEQ ID NO: 80. Another example protein that comprises a WiChR variant comprises the amino acid sequence set forth in SEQ ID NO: 82. Another example protein that comprises a WiChR variant comprises the amino acid sequence set forth in SEQ ID NO: 84. Another example protein that comprises a WiChR variant comprises the amino acid sequence set forth in SEQ ID NO: 86. Another example protein that comprises a WiChR variant comprises the amino acid sequence set forth in SEQ ID NO: 88. Another example protein that comprises a WiChR variant comprises the amino acid sequence set forth in SEQ ID NO: 90. Another example protein that comprises a WiChR variant comprises the amino acid sequence set forth in SEQ ID NO: 92. Another example protein that comprises a WiChR variant comprises the amino acid sequence set forth in SEQ ID NO: 94. Another example protein that comprises a WiChR variant comprises the amino acid sequence set forth in SEQ ID NO: 96. Another example protein that comprises a WiChR variant comprises the amino acid sequence set forth in SEQ ID NO: 98. Another example protein that comprises a WiChR variant comprises the amino acid sequence set forth in SEQ ID NO: 100. Another example protein that comprises a WiChR variant comprises the amino acid sequence set forth in SEQ ID NO: 102. Another example protein that comprises a WiChR variant comprises the amino acid sequence set forth in SEQ ID NO: 104. Another example protein that comprises a WiChR variant comprises the amino acid sequence set forth in SEQ ID NO: 106. Another example protein that comprises a WiChR variant comprises the amino acid sequence set forth in SEQ ID NO: 108. Another example protein that comprises a WiChR variant comprises the amino acid sequence set forth in SEQ ID NO: 110. Another example protein that comprises a WiChR variant comprises the amino acid sequence set forth in SEQ ID NO: 112. Another example protein that comprises a WiChR variant comprises the amino acid sequence set forth in SEQ ID NO: 114. Another example protein that comprises a WiChR variant comprises the amino acid sequence set forth in SEQ ID NO: 116. Another example protein that comprises a WiChR variant comprises the amino acid sequence set forth in SEQ ID NO: 118. Another example protein that comprises a WiChR variant comprises the amino acid sequence set forth in SEQ ID NO: 120. Another example protein that comprises a WiChR variant comprises the amino acid sequence set forth in SEQ ID NO: 122. Another example protein that comprises a WiChR variant comprises the amino acid sequence set forth in SEQ ID NO: 124. Another example protein that comprises a WiChR variant comprises the amino acid sequence set forth in SEQ ID NO: 126. Another example protein that comprises a WiChR variant comprises the amino acid sequence set forth in SEQ ID NO: 128. Another example protein that comprises a WiChR variant comprises the amino acid sequence set forth in SEQ ID NO: 130.

Various example genetic constructs that include a nucleic acid that encodes a WiChR variant are described herein. An example genetic construct comprises a nucleic acid that encodes a WiChR variant according to an embodiment.

Various example fusion proteins that include a WiChR variant are described herein. An example fusion protein comprises a WiChR variant according to an embodiment.

Various example compositions that include a nucleic acid that encodes a WiChR variant are described herein. An example composition comprises a nucleic acid that encodes a WiChR variant according to an embodiment packaged into a delivery vector, such as an adeno-associated virus. Another example composition comprises a genetic construct that includes a nucleic acid that encodes a WiChR variant according to an embodiment packaged into a delivery vector, such as an adeno-associated virus

Various example methods of treatment are described herein.

An example method of treatment comprises delivering a WiChR variant according to an embodiment to a patient in need thereof.

Another example method of treatment comprises a method of restoring vision in a patient having a form of blindness that involves degeneration of photoreceptors. The method comprises introducing one or more WiChR variants according to one or more embodiments into remaining cells in the retina via gene therapy to restore photosensitivity.

Additional understanding of the claimed WiChR variants, nucleic acids that encode a WiChR variant, genetic constructs that include a nucleic acid encoding a WiChR variant, compositions that include a nucleic acid encoding a WiChR variant, and methods of treatment can be obtained by reviewing the detailed description of selected examples, below, with reference to the appended drawings.

### BRIEF DESCRIPTION OF THE DRAWINGS

FIG. 1 is a schematic representation of the membrane topology of wild type WiChr channelrhodopsin from Wobblia lunata. The wild type sequence illustrated in the Figure is presented as SEQ ID NO: 2.

FIG. 2 is a graphical representation of results of voltage clamp experiments measuring I-V curves for the photocurrent both in wildtype WiChR and in a WiChR variant containing the F240A mutation.

FIG. 3 is a graphical representation of the reversal potential for potassium in wildtype WiChR and in a WiChR variant containing the F240A mutation.

FIG. 4 is a graphical representation of whole-cell voltage clamp recordings from HEK293T cells which were transfected with plasmid DNA encoding wildtype WiChR (left) and WiChR K204A/M219C/F240A/F245L (right).

FIG. 5 is a graphical representation of results of a high-throughput assay comparing wildtype WiChR (left panels) and WichR K204A/M219C/F240A/F245L (right panels) across a series of seven different wavelengths of light (440 nm, 475 nm, 510 nm, 555 nm, 575 nm, 637 nm, and 748 nm) at various irradiances.

FIG. 6 is a graphical representation of median photocurrent responses of wildtype WiChR and several WiChR variants to different irradiances of 475 nm light.

FIG. 7 is a graphical representation of dose-response curves for WiChR and the WiChR variants represented in FIG. 6.

FIG. 8 illustrates various summary metrics for WiChR and the WiChR variants represented in FIG. 6.

FIG. 9 illustrates a graphical representation of results of manual voltage clamp recordings taken from HEK293T cells transfected with the specified opsin after having been dark-adapted prior to recording.

FIG. 10 illustrates a graphical representation of a quantification of the steady state responses of the recordings illustrated in FIG. 9.

FIG. 11 illustrates a graphical representation of a voltage clamp recording from cells transfected with WiChR M219L/F240A (top panel) and ChReef (bottom panel) over time. The yellow arrows in each panel represent a point in time at which the relevant cells were exposed to a flashlight stimuli.

FIG. 12 illustrates a graphical representation of a voltage clamp recording of a human neuron that expresses WiChR M219L/F240A. Over time, the recording was performed on the neuron in the dark, while the neuron was exposed to an ambient light stimulus, and again in the dark.

FIG. 13 illustrates graphical representations of average traces in voltage clamp recordings of the response to strong (~1-2 mW/mm²) 475 nm light for a series of WiChR variants characterized as having accelerated kinetics.

FIG. 14 illustrates graphical representations of average traces in voltage clamp recordings of the response to strong (~1-2 mW/mm²) 475 nm light for a series of WiChR variants characterized as having decelerated kinetics.

FIG. 15 illustrates summary metrics for example WiChR variants characterized as having accelerated and decelerated kinetics.

FIG. 16 is a graphical representation of a dot plot of opsin kinetics and sensitivity parameters for several WiChR variants according to embodiments. WiChR variants according to specific embodiments are highlighted, including WiChR F240A for comparison.

FIG. 17 is a schematic illustration of a genetic construct according to an embodiment.

FIG. 18 is a schematic illustration of a genetic construct according to an embodiment.

FIG. 19 is a schematic illustration of a fusion protein according to an embodiment.

FIG. 20 is a schematic illustration of a genetic construct payload for inclusion in an adeno-associated virus in a composition according to an embodiment.

FIG. 21A illustrates a graphical representation of the maximum amplitude photocurrent (Amp Max (pA)) observed for each of several truncated variants of the S122L/F240A WiChR variant presented as SEQ ID NO: 30 and a corresponding LucyRho version (LR+) of the respective variant relative to the S122L/F240A WiChR variant.

FIG. 21B illustrates a graphical representation of the estimated peak of the action spectrum (Lambda Max (nm)) observed for each of several truncated variants of the S122L/F240A WiChR variant presented as SEQ ID NO: 30 and a corresponding LucyRho version (LR+) of the respective variant relative to the S122L/F240A WiChR variant.

FIG. 21C illustrates a graphical representation of the estimated amount of irradiance required to evoke a 200 pA photocurrent at the optimal wavelength for photostimulation (200 pA threshold (mW/mm²)) observed for each of several truncated variants of the S122L/F240A WiChR variant presented as SEQ ID NO: 30 and a corresponding LucyRho version (LR+) of the respective variant relative to the S122L/F240A WiChR variant.

FIG. 21D illustrates a graphical representation of the time constant of the decay of the photocurrent after cessation of the photostimulation (Tau off (ms)) observed for each of several truncated variants of the S122L/F240A WiChR variant presented as SEQ ID NO: 30 and a corresponding LucyRho version (LR+) of the respective variant relative to the S122L/F240A WiChR variant.

### DETAILED DESCRIPTION OF SELECTED EXAMPLES

The following detailed description and the appended drawings describe and illustrate various example channelrhodopsins, nucleic acids that encode a channelrhodopsin, genetic constructs that include a nucleic acid encoding a channelrhodopsin, compositions that include a nucleic acid encoding a channelrhodopsin, and methods of treatment. The description and drawings are provided to enable one skilled in the art to make and use one or more example channelrhodopsins, nucleic acids, genetic constructs, and compositions, and to perform one or more example methods of treatment. They are not intended to limit the scope of the claims in any manner.

As used herein, the term “about” provides literal support for the exact numerical value it precedes, as well as values approximately equal to that number. The determination of what constitutes an approximate value depends on the specific context and is intended to cover values that are substantially equivalent to the explicitly recited number in terms of function or effect.

As used herein, the singular forms “a,” “an,” and “the” include plural referents unless explicitly stated otherwise. The claims may also be drafted to exclude any optional element. This statement serves as an antecedent basis for the use of exclusive terminology such as “solely,” “only,” or similar limiting language in connection with claim elements or negative limitations.

As used herein, the terms “evaluating,” “determining,” “measuring,” “assessing,” and “assaying” are used interchangeably and refer to both qualitative and quantitative assessments.

As used herein, the terms “excitation maximum” and “absorption maximum” refer to the peak absorption wavelength of a given moiety. If a moiety is fluorescent (i.e., capable of emitting photons or other radiation upon excitation), these terms may be used interchangeably. In the field of rhodopsins, it is common to draw a distinction between an ‘action spectrum’ and an ‘absorption spectrum’, with the former being the relationship between wavelength and photocurrent. Data presented herein relates primarily to action spectrum.

As used herein, the term “light-gated” refers to the modulation of a system, process, or device through the use of light (typically in the form of photons). This includes, but is not limited to, applications such as optical switches, sensors, detectors, and other technologies where light serves as a control mechanism to trigger specific actions or responses.

As used herein, the term “nucleic acid” refers to a polymeric sequence of nucleotides of any length, including oligonucleotides (e.g., 2-100 nucleotides) and polynucleotides (e.g., greater than 50 nucleotides). A “nucleotide” consists of a sugar, a nucleobase, and a phosphate group. The terms “nucleobase” and “base” are used interchangeably. The term “nucleic acid” encompasses polymers composed of canonical nucleotides-adenine (A), cytosine (C), guanine (G), thymine (T), and uracil (U)—as well as non-canonical nucleotides, chemically or biochemically modified nucleotides, and nucleotides with non-conventional sugar-phosphate backbones. Conventional sugar-phosphate backbones include ribose-phosphate (as in ribonucleic acid (RNA)) and deoxyribose-phosphate (as in deoxyribonucleic acid (DNA)). Non-conventional backbones include, but are not limited to, backbones incorporating sugars other than ribose or deoxyribose (e.g., threose), peptide linkages, or synthetic scaffolds. Examples of non-natural or synthetic nucleic acid backbones include xeno nucleic acid (XNA), peptide nucleic acid (PNA), morpholino nucleic acid, locked nucleic acid (LNA), glycol nucleic acid (GNA), 1,5-anhydrohexitol nucleic acid (HNA), cyclohexene nucleic acid (CeNA), fluoro arabino nucleic acid (FANA), and threose nucleic acid (TNA). Hybrid nucleic acids containing a combination of conventional and non-conventional nucleotides are also encompassed. For example, a nucleic acid may contain one or more LNA-modified nucleotides interspersed within a DNA sequence. A nucleic acid may have any length suitable for its intended function, such as at least 2 nucleotides, 4 or more nucleotides, 10 or more nucleotides, 20 or more nucleotides, 50 or more nucleotides, 100 or more nucleotides, 300 or more nucleotides, up to 500, 1000, or more nucleotides.

As used herein, the term “percent identity” refers to a percentage of identity between two referenced nucleotide sequences. The percent identity of two nucleotide sequences is determined by aligning the referenced sequences for optimal comparison (e.g., introducing gaps if necessary). The percent identity is calculated as follows:

% identity=(#of identical positions/total #of positions)×100
where an identical position is one in which the nucleotide in one sequence is the same as the nucleotide in the corresponding position of the other sequence. A non-limiting example of a sequence comparison algorithm is described in Karlin et al., Proc. Natl. Acad. Sci. USA 90:5873-5877 (1993). This algorithm is implemented in the NBLAST and XBLAST programs (version 2.0), as described in Altschul et al., Nucleic Acids Res. 25:389-3402 (1997). Unless otherwise specified, the default parameters of the respective BLAST programs (e.g., NBLAST) may be used for sequence comparison. In some embodiments, parameters may be set at a score threshold of 100 and a word length of 12, with optional variations such as a word length of 5 or 20.

As used herein, the term “photocurrent” refers to the electrical current across the cell membrane induced by light stimulation. In this context, photocurrent is typically measured in picoamperes (pA) or nanoamperes (nA) and is achieved by the translocation of ions across the membrane by light-gated ion channels.

As used herein, the term “plurality” refers to a quantity of at least two members. In some embodiments, a plurality may comprise 5 or more, 10 or more, 50 or more, 100 or more, 1000 or more, 10,000 or more, or up to 100,000 or more members.

Methods described herein may include multiple steps performed sequentially or with an intervening delay. The interval between steps may range from immediate execution upon completion of the preceding step to predefined waiting periods. In some embodiments, the waiting period between steps is at least 1 second, 10 seconds, 30 seconds, 60 seconds, 5 minutes, 10 minutes, 60 minutes, or 5 hours. In other embodiments, subsequent steps are performed immediately after the completion of a prior step or after an incubation or waiting period of a few minutes to overnight.

Where a range of values is provided, it is understood that each intervening value within the stated range—including values to the nearest tenth of the unit of the lower limit, unless the context clearly dictates otherwise—is encompassed within the described methods and systems. The upper and lower limits of the range, as well as any intermediate values, are included within the scope of the disclosure unless explicitly stated otherwise. Additionally, where a range includes one or both of its endpoints, variations excluding either or both endpoints are also encompassed within the disclosure.

Unless otherwise defined, all technical and scientific terms used herein have the same meaning as commonly understood by those of ordinary skill in the relevant art. While methods and systems similar or functionally equivalent to those described herein may be used in the practice or testing of the disclosed methods and systems, representative and illustrative embodiments are described below.

All publications and patents cited in this specification are incorporated by reference as if each individual publication or patent were explicitly set forth herein. The incorporation by reference applies to the disclosure of materials and/or methods in connection with which the referenced documents are cited. The citation of any publication is solely for disclosure purposes and does not constitute an admission that the present methods and systems lack priority over the cited reference. Furthermore, the actual publication date of any referenced document may require independent verification.

For clarity, certain features of the disclosed methods and systems are described separately in distinct embodiments. However, it is understood that these features may also be combined into a single embodiment. Conversely, features that are described together in a single embodiment may also be provided separately or in any operable sub-combination. All combinations and sub-combinations of the disclosed embodiments are expressly included within the scope of the present disclosure, provided they result in an operable method or system.

As will be apparent to those skilled in the art upon reading this disclosure, each of the embodiments described herein comprises distinct components and features that may be separated or combined without departing from the scope or spirit of the invention. Any described method may be carried out in the order of steps explicitly recited or in any logically permissible order unless otherwise stated.

Channelrhodopsins (ChRs) are light-gated ion channel proteins found in certain algae. ChRs function as photoreceptors to help the host organism regulate movement in response to light. When exposed to specific wavelengths of light, ChRs open and allow ions to pass through the cell membrane, altering the electrical state of the cell. ChRs exist in multiple forms in nature, including variants that specifically conduct anions (anion channelrhodopsins, ACRs) and cations (cation channelrhodopsins, CCRs). While CCRs have been used to stimulate neuronal activity and ACRs to inhibit it, their utility is constrained by functional limits, such as the need for high-powered photostimulation. The usefulness of ACRs is further limited by the fact that, in some contexts, such as during development and certain pathological states, chloride flux is excitatory rather than inhibitory. Recently, potassium-selective channelrhodopsins (KCRs) have been described, which provide a potentially superior tool for neuronal silencing.

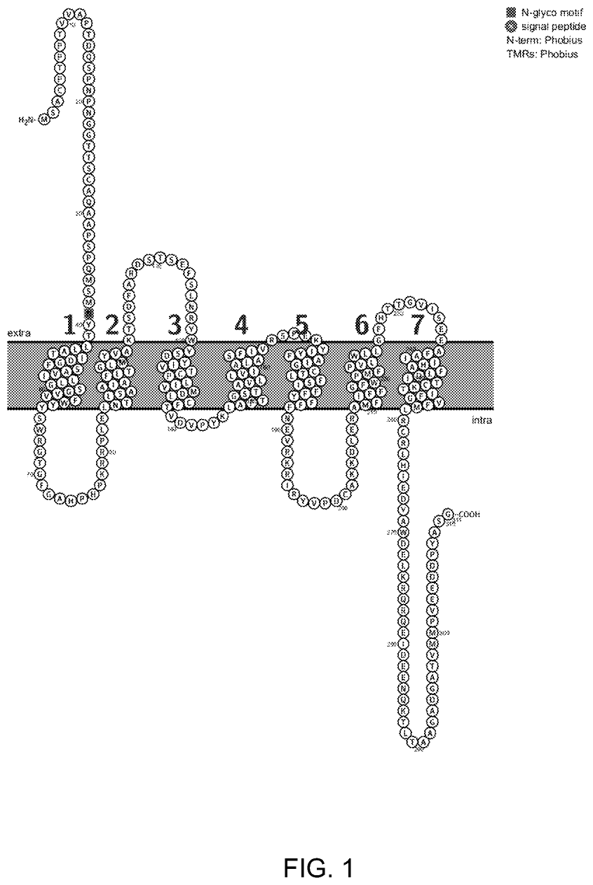

WiChR is a ChR that occurs naturally in Wobblia lunata, a microscopic marine flagellate. WiChR is sensitive and highly selective for potassium cations (WiChR is a KCR). Like other ChRs, WiChR has 7 helical transmembrane (TM) domains. The N-terminus domain preceding the first TM domain faces the extracellular space and is likely disordered. FIG. 1 illustrates the membrane topology of wild type WiChR.

The inventors have developed new excitatory optogenetic tools derived from WiChR. These engineered WiChR variants have been converted from inhibitory KCRs into excitatory CCRs. They are highly sensitive and efficacious, and have therapeutic applications for vision restoration. Embodiments of the invention relate to nucleotide sequences that encode WiChR variants, amino acid sequences for WiChR variants, genetic constructs that include a nucleotide sequence that encodes a WiChR variant, fusion proteins that include a WiChR variant, compositions that include a nucleic acid construct that encodes a WiChR variant and/or that includes a WiChR variant, and methods, such as methods of treatment that include delivering a nucleic acid construct that encodes a WiChR variant and/or a WiChR variant to a patient in need thereof.

Several embodiments described herein are directed to respective nucleotide sequences or amino acid sequences derived from corresponding naturally occurring ChR sequences. For example, Several embodiments described herein are directed to respective nucleotide sequences or amino acid sequences derived from WiChR. The nucleic acid that encodes wild type WiChR was first described in 2022 (Vierock et al., WiChR, a highly potassium-selective channelrhodopsin for low-light one- and two-photon inhibition of excitable cells; Sci Adv. 2022 Dec. 9; 8(49): eadd7729; doi:10.1126/sciadv.add7729) and is set forth in SEQ ID NO: 1. The amino acid sequence for wild type WiChR is set forth in SEQ ID NO: 2.

### Nucleic Acids and Amino Acids

Several embodiments are directed to nucleic acids that encode a ChR according to an embodiment. As such, nucleic acid embodiments can comprise a nucleic acid coding sequence that encodes a ChR explicitly described herein or that is within the scope of embodiments of ChRs described herein. A coding sequence in a nucleic acid according to an embodiment can include one or more exons and zero or more introns and can be optimized for expression in a particular species. Nucleic acids according to embodiments can have any suitable form, including linear, circular, and any suitable strandedness, including single stranded, and double stranded, for any particular host species and/or purpose. Also, nucleic acids according to embodiments can be incorporated into genetic constructs according to embodiments, as described in detail below. Also, while DNA sequences are described herein, it is understood that corresponding RNA sequences are contemplated by the inventors and are considered within the scope of the invention. RNA sequences may have individual utility beyond the utility of DNA sequences.

Nucleic acids according to embodiments can be synthesized using any suitable techniques, procedures, and processes, such as solid-phase synthesis, enzymatic synthesis, such as reverse transcription using reverse transcriptase to generate complementary DNA (cDNA) from an RNA template. Polymerase chain reaction (PCR) and polymerase cycling assembly (PCA) techniques can also be used in synthesis of nucleic acids according to embodiments.

Once synthesized, nucleic acids according to embodiments can be further processed and/or treated using one or more suitable techniques, procedures, and processes for purification, quality assessment, and quantification. For example, nucleic acids according to embodiments can be subjected to high-performance liquid chromatography (HPLC), ultrafiltration, size-exclusion chromatography, or other suitable techniques for removing contaminants. Quality and quantity assessments can be conducted using spectroscopy, electrophoresis, or other analytical methods to ensure integrity and consistency of the synthesized nucleic acids according to embodiments.

Vierock et al. (supra) identified residues in the ion pore domain of WiChR that are important for its selectivity to potassium. These residues include D47, N117, W120, and F240. Homologous residues have been studied in two other potassium selective channelrhodopsins, HcKCR1 and HcKCR2 (Govorunova et al. 2022; Govorunova et al. 2022; Zhang et al. 2022; Tajima et al. 2023).

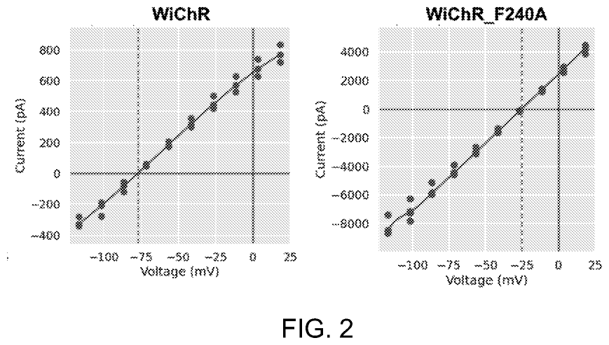

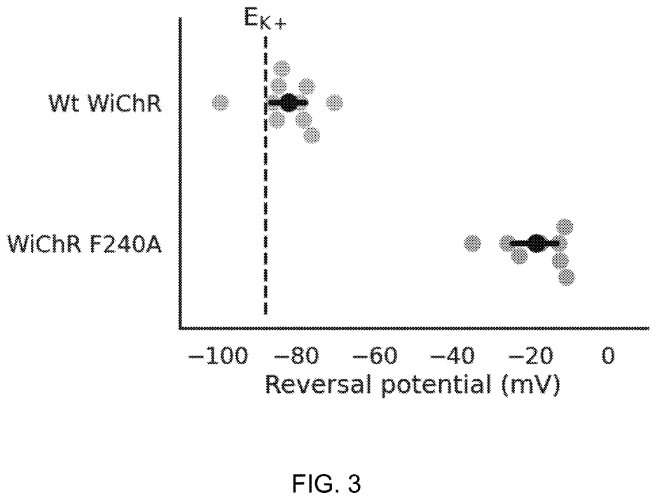

The inventors have identified mutations and combinations of mutations in wild type WiChR that reduce or abolish the ChRs selectivity for potassium ions. Functionally, these mutations convert WiChR from an inhibitory optogenetic tool into an excitatory optogenetic tool. In one embodiment, a point mutation is introduced at residue 240, changing Phenylalanine to Alanine. This mutation is designated as F240A, and the WiChR derivative having this mutation is designated as WiChR F240A. The inventors also refer to this WiChR derivative as WAChR. As shown in FIGS. 2 and 3, which illustrate results of voltage clamp experiments which measure I-V curves for the photocurrent both in wildtype WiChR and in a WiChR variant containing the F240A mutation, the reversal potential for wildtype WiChr is-85 mV (close to the calculated reversal potential for potassium in these conditions) whereas the reversal potential for the WiChR F240A photocurrent is depolarized to roughly-25 mV, reflecting a loss of selectivity for potassium.

The DNA sequence set forth in SEQ ID NO: 3 encodes WiChR F240A. The amino acid sequence set forth in SEQ ID NO: 4 is the resulting protein sequence for WiChR F240A.

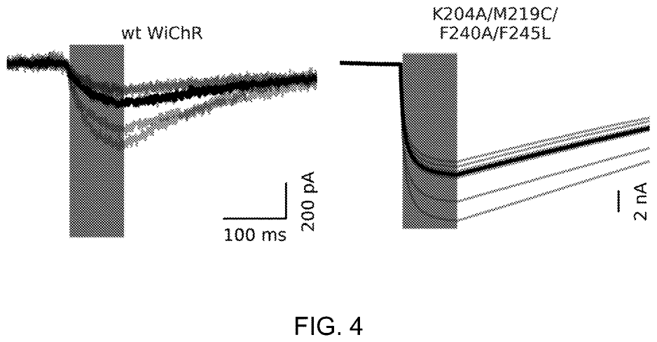

Importantly, the F240A mutation, which is a point mutation, preserves other important functional properties of WiChR and is readily combined with other mutations, as described below. Thus, WiChR F240A represents a foundational mutation for the embodiments described herein. Indeed, the inventors have determined that the F240A mutation, combined with other mutations from the wild type WiChR sequence, can enhance the functional properties of WiChR F240A and/or provide additional desirable properties to the resulting protein. For example, the inventors have determined that a modified WiChR, designated as WiChR K204A/M219C/F240A/F245L, along with three other point mutations-K204A (Lysine to Alanine at residue 204), M219C (Methionine to Cysteine at residue 219), and F245L Phenylalanine to Leucine at residue 245)-demonstrates much larger photocurrents that wild type WiChR. FIG. 4 illustrates results of whole-cell voltage clamp recordings from HEK293T cells which were transfected with plasmid DNA encoding wildtype WiChR (left) and WichR K204A/M219C/F240A/F245L (right) in response to illumination with a strong blue light stimulus (475 nm, 1.53 mW/mm² and 1.22 mW/mm² on left and right panels respectively). Gray traces in each panel represent individual trials and the thick black line in each panel represents the median response. The observed shift is likely partly due to the increased driving force from the F240A mutation.

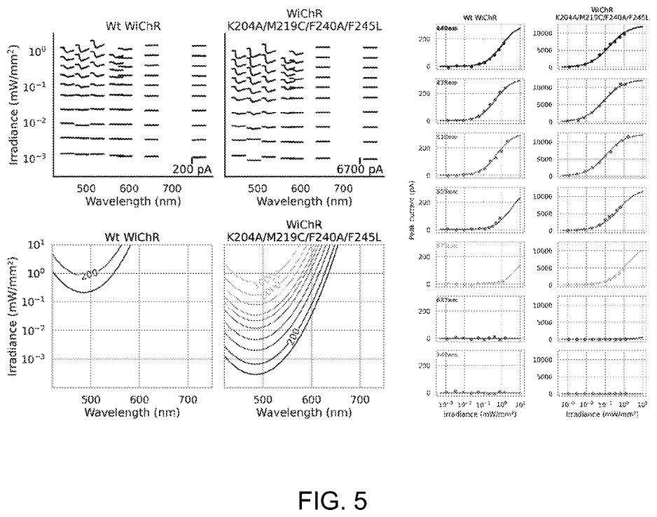

The inventors developed a high-throughput assay to assess photocurrent at combinations of wavelength and irradiance and any dose-response relationship between irradiance and the peak photocurrent across a series of seven different wavelengths of light (440 nm, 475 nm, 510 nm, 555 nm, 575 nm, 637 nm, and 748 nm). FIG. 5 illustrates the results of this high throughput assay comparing wild type WiChR and WichR K204A/M219C/F240A/F245L. As illustrated in FIG. 5, WiChR K204A/M219C/F240A/F245L exhibits much larger photocurrents than wild type WiChR. This is partly due to the increased driving force from the F240A mutation, as described above. However, dose-response curves for WiChR K204A/M219C/F240A/F245L, illustrated on the right side of FIG. 5, are shifted to the left relative to wildtype WiChR across all responsive wavelengths, reflecting increased global sensitivity from its mutations other than the F240A mutation. This can also be seen in the bottom left panels, which show a continuous estimate of the peak photocurrent response as a contour map in wavelength-irradiance space.

The DNA sequence set forth in SEQ ID NO: 5 encodes WiChR K204A/M219C/F240A/F245L. The amino acid sequence set forth in SEQ ID NO: 6 is the resulting protein sequence for WiChR K204A/M219C/F240A/F245L.

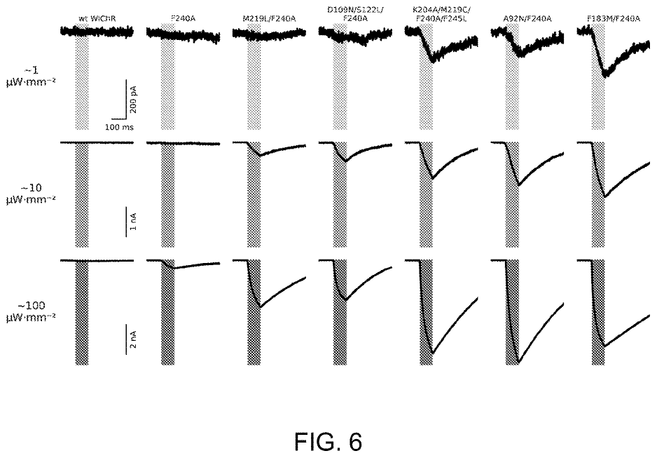

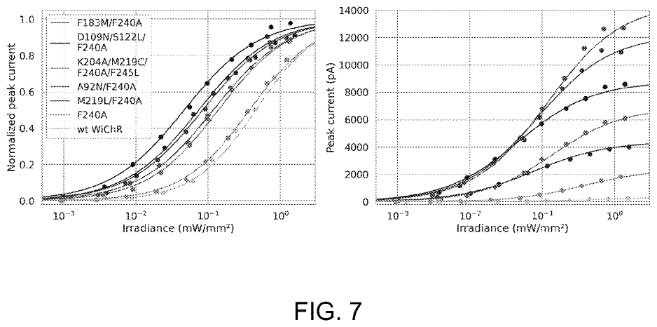

The inventors developed a high-throughput assay with photocurrent traces and dose-response curves, reflective of the assay that provided the results for wild type WiChR and WiChR K204A/M219C/F240A/F245L illustrated in FIG. 5, to efficiently screen for functionally enhanced WiChR variants that demonstrate desirable properties similar to WiChR K204A/M219C/F240A/F245L. Experiments using this high-throughput assay evaluated several WiChR variants, including WiChR M219L/F240A (Methionine to Leucine at residue 219, Phenylalanine to Alanine at residue 240), WiChR D109N/S122L/F240A (Aspartic Acid to Asparagine at residue 109, Serine to Leucine at residue 122, Phenylalanine to Alanine at residue 240), WiChR A92N/F240A (Alanine to Asparagine at residue 92, Phenylalanine to Alanine at residue 240), and WiChR F183M/F240A (Phenylalanine to Methionine at residue 183, Phenylalanine to Alanine at residue 240). FIG. 6 illustrates results of this high throughput assay, showing median photocurrent responses for each these variants, wild type WiChR, and WichR K204A/M219C/F240A/F245L, to different irradiances of 475 nm light. FIG. 7 illustrates the dose-response curves for wild type WiChR and the WiChR variants represented in FIG. 6.

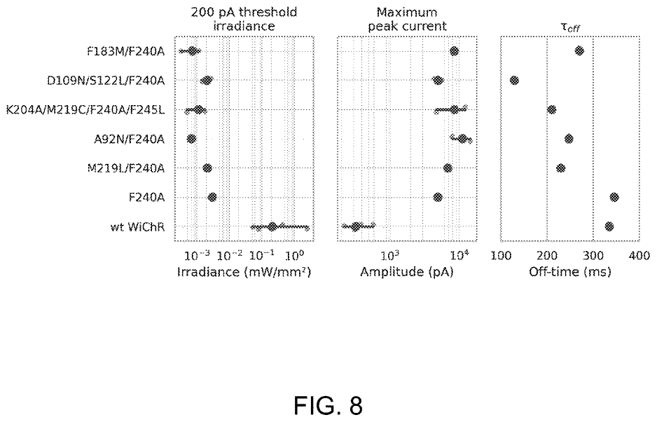

FIG. 8 illustrates various summary metrics of these WiChR variants alongside wild type WiChR and WichR F240A and WichR WiChR K204A/M219C/F240A/F245L. The first panel illustrates 200 pA threshold irradiance of the WiChR variants as a summary metric of the sensitivity of the WiChR variants, the second panel illustrates estimates of maximum peak current as a summary metric of the efficacy of the WiChR variants at saturating irradiances, and the third panel illustrates an off-time constant as a summary metric of the kinetics of the WiChR variants.

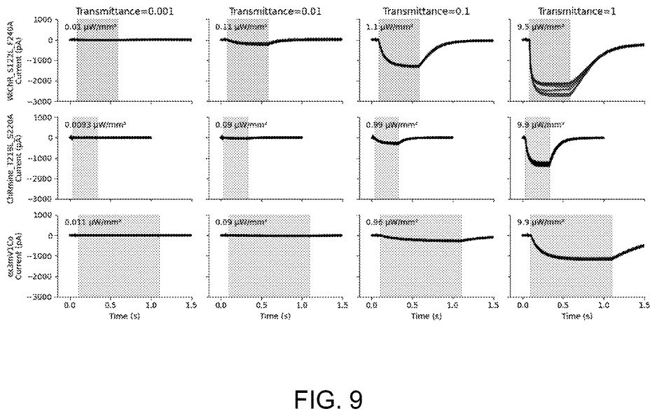

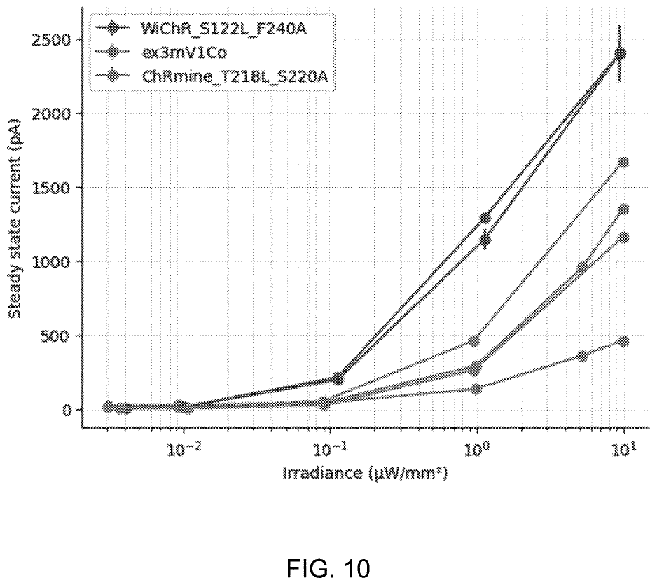

To benchmark the low-light sensitivity of an example WiChR variant, the inventors compared photocurrent responses of WiChR S122L/F240A (Serine to Leucine at residue 122, Phenylalanine to Alanine at residue 240) to existing highly sensitive opsins, ChRmine T218L/S220A (“ChReef”; Zerche et al. 2023) and ex3m1VCo (Watanabe et al. 2021) during photostimulation with varying irradiances at a wavelength close to each opsin's spectral peak (480 nm for WiChR S122L/F240A, 520 nm for ChReef and ex3m1VCo). FIG. 9 illustrates manual voltage clamp recordings taken from HEK293T cells transfected with these opsins after having been dark-adapted prior to recording. FIG. 10 illustrates a quantification of the steady state responses of these recordings. As illustrated in FIG. 10, WiChR S122L/F240A first exhibits robust photocurrents at irradiances on the order of 0.1 uW/mm² whereas the other opsins require irradiances on the order of 1 pW/mm².

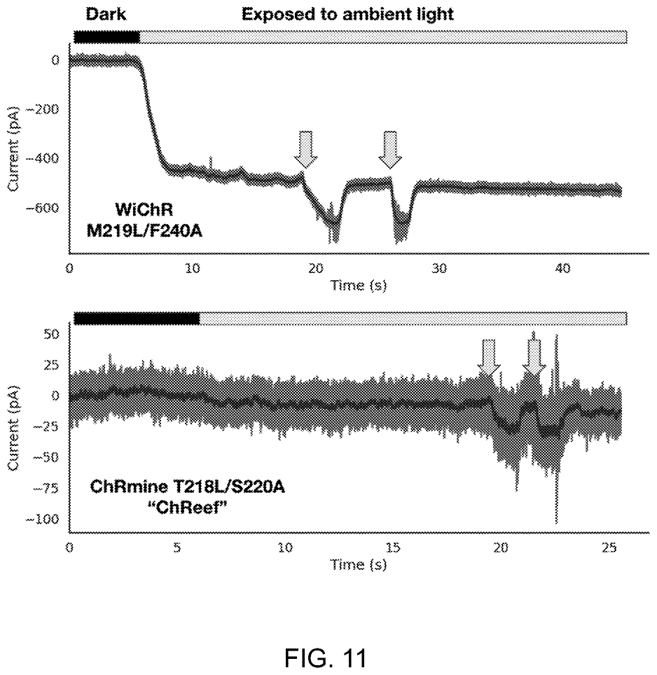

WiChR variants according to some embodiments are sufficiently sensitive to be responsive to ambient indoor light. FIG. 11 illustrates results of an experiment in which transfected HEK293T cells are dark-adapted, and then exposed to ambient light (by opening a curtain enclosing a patch clamp apparatus). The top panel in FIG. 11 shows a voltage clamp recording from cells transfected with WiChR M219L/F240A (Methionine to Leucine at residue 219, Phenylalanine to Alanine at residue 240). Ambient light evokes currents of roughly 500 pA in this cell. In FIG. 11, the yellow arrows indicate points in time where an experimenter pointed a cell phone flashlight at the recording stage. This experiment, while obviously crude, demonstrates that WiChR M219L/F240A is readily activated by household light sources. Conversely, as illustrated in the bottom panel in FIG. 11, no detectable response to ambient light was observed in cells transfected with ChReef, and only a small response (<25 pA) to the flashlight stimuli (again indicated by the yellow arrows).

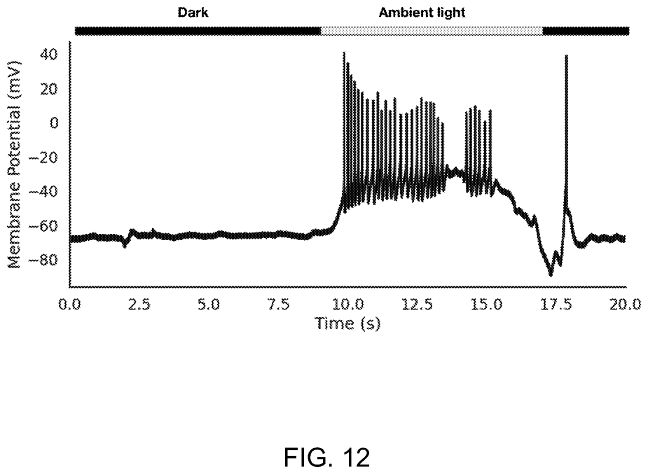

FIG. 12 illustrates results of an experiment analogous to the experiment reflected in FIG. 11. In this experiment, however, the recording reflects the current-clamp configuration of a human neuron which was derived from an induced pluripotent stem cell and made to express WiChR M219L/F240A via infection with an adeno associated virus (AAV) containing a nucleic acid encoding WiChR M219L/F240A. As illustrated in FIG. 12, exposing this neuron to light is sufficient to drive sustained depolarization and firing of action potentials, which cease when the stimulus is removed.

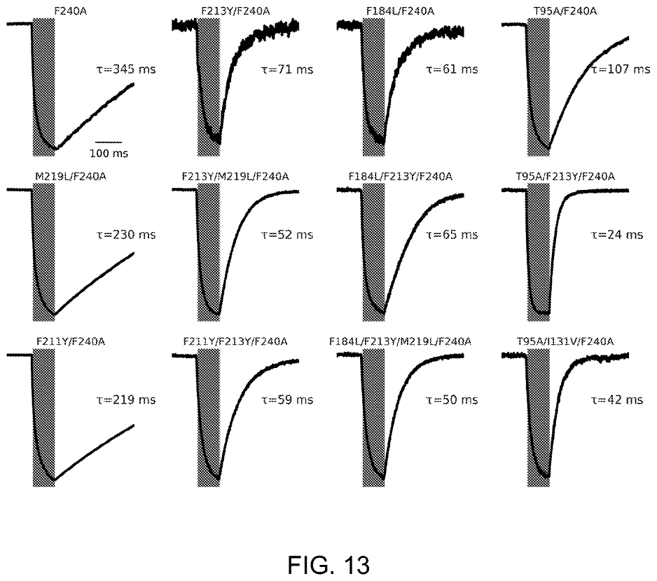

The inventors have identified WiChR variants that accelerate the kinetics of WiChR. For example, FIG. 13 illustrates average traces in voltage clamp recordings of the response to strong (~1-2 mW/mm²) 475 nm light for a series of WiChR variants characterized as having accelerated kinetics, including WiChR F213Y/F240A (Phenylalanine to Tyrosine at residue 213, Phenylalanine to Alanine at residue 240), WiChR F184L/F240A (Phenylalanine to Leucine at residue 184, Phenylalanine to Alanine at residue 240), WiChR T95A/F240A (Threonine to Alanine at residue 95, Phenylalanine to Alanine at residue 240), WiChR M219L/F240A (Methionine to Leucine at residue 219, Phenylalanine to Alanine at residue 240), WiChR F213Y/M219L/F240A (Phenylalanine to Tyrosine at residue 213, Methionine to Leucine at residue 219, Phenylalanine to Alanine at residue 240), WiChR F184L/F213Y/F240A (Phenylalanine to Leucine at residue 184, Phenylalanine to Tyrosine at residue 213, Phenylalanine to Alanine at residue 240), WiChR T95A/F213Y/F240A (Threonine to Alanine at residue 95, Phenylalanine to Tyrosine at residue 213, Phenylalanine to Alanine at residue 240), WiChR F211Y/F240A (Phenylalanine to Tyrosine at residue 211, Phenylalanine to Alanine at residue 240), WiChR F211Y/F213Y/F240A (Phenylalanine to Tyrosine at residue 211, Phenylalanine to Tyrosine at residue 213, Phenylalanine to Alanine at residue 240), WiChR F184L/F213Y/M219L/F240A (Phenylalanine to Leucine at residue 184, Phenylalanine to Tyrosine at residue 213, Methionine to Leucine at residue 219, Phenylalanine to Alanine at residue 240), and WichR T95A/1131V/F240A (Threonine to Alanine at residue 95, Isoleucine to Valine at residue 131, Phenylalanine to Alanine at residue 240). WiChR F240A is included for comparison.

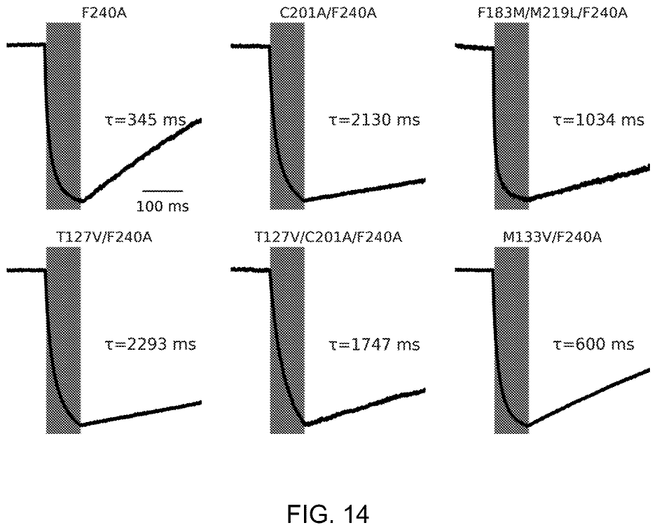

Similarly, the inventors have identified WiChR variants that decelerate the kinetics of WiChR. For example, FIG. 14 illustrates average traces in voltage clamp recordings of the response to strong (~1-2 mW/mm²) 475 nm light for a series of WiChR variants characterized as having decelerated kinetics, including WiChR C201A/F240A (Cysteine to Alanine at residue 201, Phenylalanine to Alanine at residue 240), WiChR F183M/M219L/F240A (Phenylalanine to Methionine at residue 183, Methionine to Leucine at residue 219, Phenylalanine to Alanine at residue 240), WiChR T127V/F240A (Threonine to Valine at residue 127, Phenylalanine to Alanine at residue 240), WiChR T127V/C201A/F240A (Threonine to Valine at residue 127, Cysteine to Alanine at residue 201, Phenylalanine to Alanine at residue 240), and WichR M133V/F240A (Methionine to Valine at residue 133, Phenylalanine to Alanine at residue 240). WiChR F240A is included for comparison.

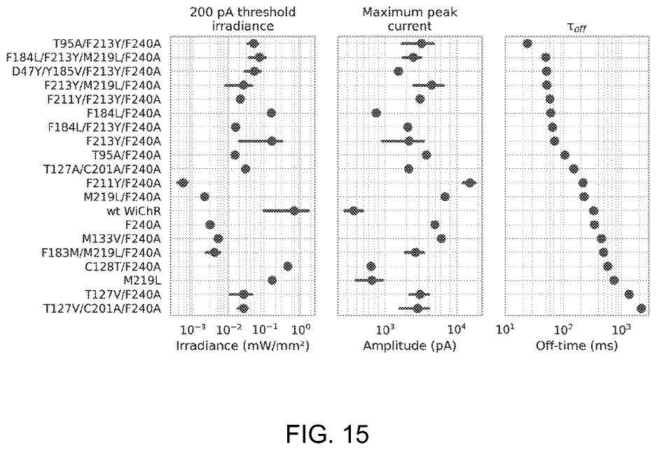

FIG. 15 illustrates summary metrics for these WiChR variants characterized as having accelerated and decelerated kinetics.

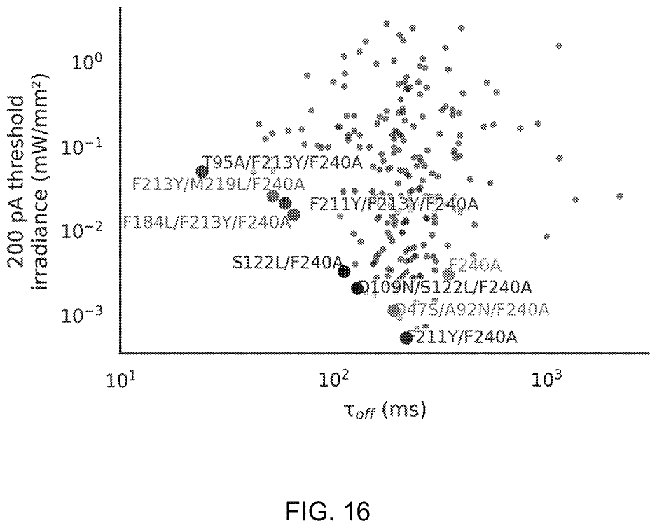

Acceleration of opsin kinetics often comes at the expense of sensitivity (Pan et al. 2014; Yan et al. 2023; Mattis et al. 2011; Berndt et al. 2011). Example WiChR variants characterized as having accelerated kinetics demonstrated this tradeoff. FIG. 16 highlights a subset of example WiChR variants according to embodiments, including WiChR T95A/F213Y/F240A, WiChR F213Y/M219L/F240A, WiChR F211Y/F213Y/F240A, WiChR F184L/F213Y/F240A, WiChR S122L/F240A, WiChRD109N/S122L/F240A, WiChR D47S/A92N/F240A, and WichR F211Y/F240A, that are at or near the Pareto frontier of these two properties. WiChR F240A is highlighted for comparison.

Nucleotide sequences and amino acid sequences for the WiChR variants according to the specific embodiments described above are included in the disclosure as follows. The DNA sequence set forth in SEQ ID NO: 7 encodes WiChR T127V/F240A. The amino acid sequence set forth in SEQ ID NO: 8 is the resulting protein sequence for WiChR T127V/F240A. The DNA sequence set forth in SEQ ID NO: 9 encodes WiChR M219L/F240A. The amino acid sequence set forth in SEQ ID NO: 10 is the resulting protein sequence for WiChR M219L/F240A. The DNA sequence set forth in SEQ ID NO: 11 encodes WiChR T127V/C201A/F240A. The amino acid sequence set forth in SEQ ID NO: 12 is the resulting protein sequence for WiChR T127V/C201A/F240A. The DNA sequence set forth in SEQ ID NO: 13 encodes WiChR C201A/F240A. The amino acid sequence set forth in SEQ ID NO: 14 is the resulting protein sequence for WiChR C201A/F240A. The DNA sequence set forth in SEQ ID NO: 15 encodes WiChR T95A/1131V/F240A. The amino acid sequence set forth in SEQ ID NO: 16 is the resulting protein sequence for WiChR T95A/1131V/F240A. The DNA sequence set forth in SEQ ID NO: 17 encodes WiChR F213Y/F240A. The amino acid sequence set forth in SEQ ID NO: 18 is the resulting protein sequence for WiChR F213Y/F240A. The DNA sequence set forth in SEQ ID NO: 19 encodes WiChR F211Y/F240A. The amino acid sequence set forth in SEQ ID NO: 20 is the resulting protein sequence for WiChR F211Y/F240A. The DNA sequence set forth in SEQ ID NO: 21 encodes WiChR L206F/F240A. The amino acid sequence set forth in SEQ ID NO: 22 is the resulting protein sequence for WiChR L206F/F240A. The DNA sequence set forth in SEQ ID NO: 23 encodes WiChR A92N/F240A. The amino acid sequence set forth in SEQ ID NO: 24 is the resulting protein sequence for WiChR A92N/F240A. The DNA sequence set forth in SEQ ID NO: 25 encodes WiChR F173Y/F240A. The amino acid sequence set forth in SEQ ID NO: 26 is the resulting protein sequence for WiChR F173Y/F240A. The DNA sequence set forth in SEQ ID NO: 27 encodes WiChR F183M/F240A. The amino acid sequence set forth in SEQ ID NO: 28 is the resulting protein sequence for WiChR F183M/F240A. The DNA sequence set forth in SEQ ID NO: 29 encodes WiChR S122L/F240A. The amino acid sequence set forth in SEQ ID NO: 30 is the resulting protein sequence for WiChR S122L/F240A. The DNA sequence set forth in SEQ ID NO: 31 encodes WiChR F183M/F211Y/F240A. The amino acid sequence set forth in SEQ ID NO: 32 is the resulting protein sequence for WiChR F183M/F211Y/F240A. The DNA sequence set forth in SEQ ID NO: 33 encodes WiChR F211Y/F213Y/F240A. The amino acid sequence set forth in SEQ ID NO: 34 is the resulting protein sequence for WiChR F211Y/F213Y/F240A. The DNA sequence set forth in SEQ ID NO: 35 encodes WiChR F213Y/M219L/F240A. The amino acid sequence set forth in SEQ ID NO: 36 is the resulting protein sequence for WiChR F213Y/M219L/F240A. The DNA sequence set forth in SEQ ID NO: 37 encodes WiChR T95A/F213Y/F240A. The amino acid sequence set forth in SEQ ID NO: 38 is the resulting protein sequence for WiChR T95A/F213Y/F240A. The DNA sequence set forth in SEQ ID NO: 39 encodes WiChR F184L/F213Y/F240A. The amino acid sequence set forth in SEQ ID NO: 40 is the resulting protein sequence for WiChR F184L/F213Y/F240A. The DNA sequence set forth in SEQ ID NO: 41 encodes WiChR D47S/A92N/F240A. The amino acid sequence set forth in SEQ ID NO: 42 is the resulting protein sequence for WiChR D47S/A92N/F240A.

Additional WiChR variants are contemplated and considered part of the invention. DNA sequences that encode example WiChR variants are set forth in odd-numbered SEQ ID NOs between SEQ ID NO: 43 and SEQ ID NO: 340. Amino acid sequences that result from these DNA sequences, each of which represents a protein of the appropriate WiChR variant, are set forth as even-numbered SEQ ID NOs between SEQ ID NO: 43 and SEQ ID NO: 340. For each odd-numbered SEQ ID NO in that range, which sets forth a DNA sequence that encodes a WiChR variant, the amino acid sequence for the resulting protein is set forth in the immediately following, even-numbered SEQ ID NO.

Furthermore, truncated versions of WiChR variants are contemplated and considered part of the invention. For example, DNA sequences that represent truncated versions of the S122L/F240A WiChR variant presented as SEQ ID NO: 29 are set forth in odd-numbered SEQ ID NOs between SEQ ID NO: 341 and SEQ ID NO: 350. Amino acid sequences that result from these DNA sequences, each of which represents a protein of the appropriate WiChR variant, are set forth as even-numbered SEQ ID NOs between SEQ ID NO: 341 and SEQ ID NO: 350. For each odd-numbered SEQ ID NO in that range, which sets forth a DNA sequence that is a truncated version of the S122L/F240A WiChR variant presented as SEQ ID NO: 29, the amino acid sequence for the resulting protein is set forth in the immediately following, even-numbered SEQ ID NO.

The DNA sequence set forth in SEQ ID NO: 341 encodes the amino acid sequence set forth in SEQ ID NO: 342, which has the first six (6) amino acids truncated from the N-terminus of the opsin domain of the S122L/F240A WiChR variant presented as SEQ ID NO: 30. The DNA sequence set forth in SEQ ID NO: 343 encodes the amino acid sequence set forth in SEQ ID NO: 344, which has the first twelve (12) amino acids truncated from the N-terminus of the opsin domain of the S122L/F240A WiChR variant presented as SEQ ID NO: 30. The DNA sequence set forth in SEQ ID NO: 345 encodes the amino acid sequence set forth in SEQ ID NO: 346, which has the first seventeen (17) amino acids truncated from the N-terminus of the opsin domain of the S122L/F240A WiChR variant presented as SEQ ID NO: 30. The DNA sequence set forth in SEQ ID NO: 347 encodes the amino acid sequence set forth in SEQ ID NO: 348, which has the first twenty-five (25) amino acids truncated from the N-terminus of the opsin domain of the S122L/F240A WiChR variant presented as SEQ ID NO: 30. The DNA sequence set forth in SEQ ID NO: 349 encodes the amino acid sequence set forth in SEQ ID NO: 350, which has the first thirty-seven (37) amino acids truncated from the N-terminus of the opsin domain of the S122L/F240A WiChR variant presented as SEQ ID NO: 30.

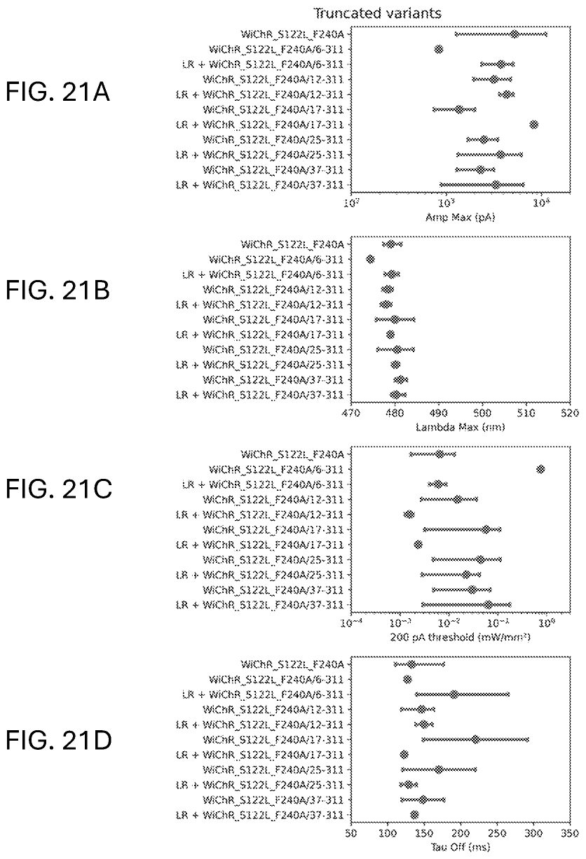

Each of the truncated amino acid sequences set forth in SEQ ID NO: 342, SEQ ID NO: 344, SEQ ID NO: 346, SEQ ID NO: 348, and SEQ ID NO: 350 retains an acceptable level of functionality. For example, each of FIGS. 21A, 21B, 21C, and 21D illustrates results of a functional assessment of each of these truncated variants, identified as WiChR S122L F240A/N-311 where N represents the position in the sequence of the S122L/F240A WiChR variant presented as SEQ ID NO: 30 of the first amino acid in the variant sequence after an initial methionine (M/Met) residue as a translation initiation signal. Thus, the WiChR S122L F240A/6-311 variant, the sequence of which is set forth in SEQ ID NO. 342, includes the sixth amino acid of SEQ ID NO. 30 as its first amino acid after an initial methionine residue. This same nomenclature also applies to each of the sequences set forth as SEQ ID NO: 344, SEQ ID NO: 346, SEQ ID NO: 348, and SEQ ID NO: 350. Each of FIGS. 21A, 21B, 21C, and 21D illustrates results for the truncated variants corresponding to the amino acid sequences presented as SEQ ID NO: 342, SEQ ID NO: 344, SEQ ID NO: 346, SEQ ID NO: 348, and SEQ ID NO: 350 and the S122L/F240A WiChR variant presented as SEQ ID NO: 30. Also, each of FIGS. 21A, 21B, 21C, and 21D illustrates results for a version of each of the truncated variants corresponding to the amino acid sequences presented as SEQ ID NO: 342, SEQ ID NO: 344, SEQ ID NO: 346, SEQ ID NO: 348, and SEQ ID NO: 350 with the LucyRho tag prepended to the N-terminus of the respective sequence (designated as “LR+” with each variant name in the Figures.) FIG. 21A shows the maximum amplitude photocurrent (Amp Max (pA)) observed for each of the truncated variants and its LucyRho version under any condition in an automated-patch clamp assay relative to the S122L/F240A WiChR variant. FIG. 21B shows the estimated peak of the action spectrum (Lambda Max (nm)) for each of the truncated variants and its LucyRho version relative to the S122L/F240A WiChR variant. FIG. 21C shows the estimated amount of irradiance required to evoke a 200 pA photocurrent at the optimal wavelength for photostimulation (200 pA threshold (mW/mm\hat{ }2)) for each of the truncated variants and its LucyRho version relative to the S122L/F240A WiChR variant. Finally, FIG. 21D shows the time constant of the decay of the photocurrent after cessation of the photostimulation (Tau off (ms)) for each of the truncated variants and its LucyRho version relative to the S122L/F240A WiChR variant. The results presented in FIGS. 21A, 21B, 21C, and 21D demonstrate that each of the truncated variants evaluated largely display unchanged functionality as compared to that of the S122L/F240A WiChR variant presented as SEQ ID NO: 30 and also referred to herein as WAChR. Furthermore, the inventors believe the proteins represented by these amino acid sequences may provide a reduced immunogenicity risk when expressed in the membranes of cells in a patient, such as via gene therapy or cell therapy, as compared to that of the full length version of the variant protein. The N-terminus of the opsin may represent a potentially high risk site for the binding of antibodies if adaptive immunity is raised against the opsin by the host. Truncation or removal of this domain may mitigate this risk, and amino acid sequences reflecting truncation or removal of the domain while also retaining an acceptable level of functionality are desirable.

The inventors believe that amino acid sequences reflecting truncation of the six (6), twelve (12), seventeen (17), twenty-five (25), or thirty-seven (37) amino acids from the N-terminus of the opsin domain of any WAChR variant within the scope of the invention, including any WiChR variant that includes the F240A point mutation, will provide a protein that retains an acceptable level of functionality. Furthermore, the inventors believe that amino acid sequences reflecting truncation of between one (1) and thirty-seven (37) amino acids from the N-terminus of the opsin domain of any WAChR variant within the scope of the invention, including any WiChR variant that includes the F240A point mutation, will provide a protein that retains an acceptable level of functionality. Truncated variations may benefit from inclusion of other N-terminus sequences like the LucyRho tag to maintain good expression and/or trafficking.

It is contemplated that the DNA and amino acid sequences disclosed herein may tolerate minor variations while retaining their functional properties. Such variations may include conservative substitutions, insertions, deletions, or modifications that do not materially alter the structure, stability, or activity of the encoded nucleic acid or protein. Accordingly, sequences having at least 70%, 75%, 80%, 85%, 90%, 91%, 92%, 93%, 94%, 95%, 96%, 97%, 98%, or 99% identity to any of the disclosed sequences are considered within the scope of the invention. Sequence identity may be determined using standard alignment algorithms such as BLAST, ClustalW, or other suitable computational methods known in the art. Variants that retain substantially equivalent biological activity, function, or expression characteristics to the disclosed sequences are encompassed by the present invention, including sequences that exhibit equivalent optogenetic, enzymatic, binding, or signaling properties.

It is further contemplated that specific point mutations disclosed herein in accordance with a sequence according to one embodiment may be combined in various permutations to generate additional sequence variants that retain the functional properties of the disclosed DNA and amino acid sequences. Accordingly, one or more point mutations described in a given sequence may be combined with one or more point mutations from another disclosed sequence to produce a variant that remains within the scope of the invention. Such combinations may result in sequences having modified but functionally equivalent characteristics, including but not limited to altered stability, expression levels, or functional activity as described herein. Variants generated by combining disclosed mutations while maintaining at least 70%, 75%, 80%, 85%, 90%, 91%, 92%, 93%, 94%, 95%, 96%, 97%, 98%, or 99% sequence identity to an original disclosed sequence are expressly encompassed within the invention.

### Genetic Constructs

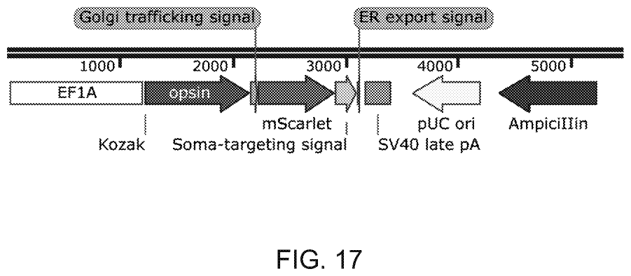

Several embodiments are directed to expression vectors that include a nucleic acids that encodes a WiChR variant according to an embodiment along with various other components. These expression vectors are useful for transfecting cells, such as HEK293T cells, that can subsequently be used in functional assays to assess the performance of the relevant WiChR variant, among other purposes. FIG. 17 illustrates a genetic construct according to an embodiment. The construct includes a nucleic acid encoding a WiChR variant according to an embodiment (“opsin”), along with a promoter. In the illustrated embodiment, the promoter comprises the ubiquitous promoter EF1a, but other suitable promoters can be used. Different promoters can be desirable in order to adjust the level of expression (e.g. a weaker promoter such as EFS), to achieve conditional expression (e.g. an inducible TRE promoter), or to target expression to specific tissues or cell types (e.g. GRM6, synapsin).

Also as illustrated in FIG. 17, genetic constructs according to embodiments can include additional optional components, such as an N-terminus signal sequence to improve membrane trafficking, one or more copies of a signal peptide to improve trafficking from the Golgi apparatus to the plasma membrane, a short, flexible peptide linker sequence (e.g. AAA), a tag or fluorescent marker protein, one or more copies of a signal peptide to induce preferential localization of the fusion protein in specific cellular compartments, such as the somatodendritic membrane compartment of neurons, and one or more copies of a signal peptide to improve export from the endoplasmic reticulum.

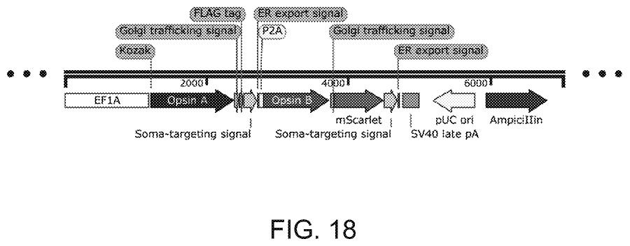

FIG. 18 illustrates a genetic construct according to an embodiment in which engineered WiChR variants are expressed via bicistronic constructs, where the coding sequences for two (or more) opsin fusion proteins are joined in a single transcript. A self-cleaving peptide (e.g., P2A, T2A, E2A) or an internal ribosome entry site (IRES) sequence ensures each opsin is translated separately, allowing co-expression in the same cell. Genetic constructs according to these embodiments can be advantageous for several reasons. First, placing two WiChR variants in a single transcript can raise the overall level of the WiChR variant in the membrane. This can boost photocurrents and improve light sensitivity without needing multiple separate expression cassettes. Also, by pairing two WiChR variants with different peak sensitivities (e.g., one blue-shifted, one red-shifted), cells can respond more efficiently to broadband or polychromatic light. In clinical scenarios, this could improve performance under normal indoor or outdoor illumination, which naturally contains many wavelengths. Furthermore, WiChR variants in a genetic construct according to a particular embodiment can be selected to balance speed and sensitivity. For instance, a slow, highly sensitive WiChR variant may respond well to very low light levels, while a faster but less sensitive WichR variant could handle high-frequency or high-intensity stimuli. Having both channels in the same cell ensures that at least one WiChR variant is optimally engaged across a broader range of light conditions. Also, in applications where the photostimulation is controlled by an external device such as a headset, co-expression of distinct WiChR variants enables more sophisticated schemes of encoding external information. For example, in the scenario where target cells express two WiChR variants with distinct spectral tuning (e.g. a blue-sensitive WiChR variant and a red-sensitive WiChR variant), intensity information from a grayscale representation of the environment could be partitioned across the two color channels. A simple example of this would be mapping the lower half of the grayscale intensity range onto levels of blue photostimulation that cover the full dynamic range of the blue opsin. Once the blue opsin saturates at moderate brightness, the display could begin to add increasing levels of red photostimulation to encode the upper half of intensities. More sophisticated schemes are possible as well.

Genetic constructs according to these embodiments can include one or more nucleic acids that encode a WiChR variant according to a first embodiment and one or more nucleic acids that encode a WiChR variant according to a second embodiment. For example, using the example illustrated in FIG. 18, “Opsin A” can comprise a nucleotide sequence that encodes a WiChR variant according to a first embodiment, such as one or the DNA sequences explicitly disclosed herein or a nucleotide sequence according to another embodiment, and “Opsin B” can comprise a nucleotide sequence that encodes a WiChR variant according to a second, different embodiment, such as one or the DNA sequences explicitly disclosed herein or a nucleotide sequence according to another embodiment. It is noted, though, that genetic constructs according to these embodiments can include multiple copies of the nucleotide sequence that encodes the same WiChR variant. For example, again with reference to the example illustrated in FIG. 18, “Opsin A” and “Opsin B” can comprises distinct copies of the same nucleotide sequence that encodes a WiChR variant according to an embodiment, such as one or the DNA sequences explicitly disclosed herein or a nucleotide sequence according to another embodiment. Also, in other embodiments, genetic constructs include a nucleic acid that encodes a WiChR variant according to an embodiment and one or more nucleic acids that encode another, non-WiChR channelrhodopsin.

### Fusion Proteins

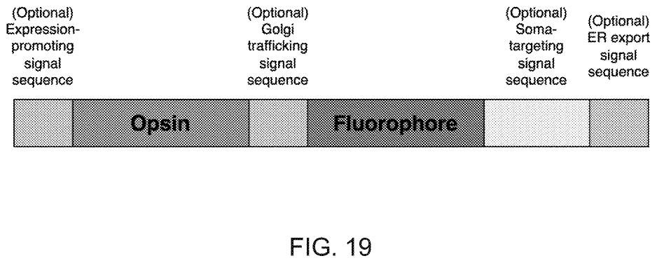

Several embodiments are directed to fusion proteins that include an amino acid sequence of a WiChR variant according to an embodiment, along with one or more additional optional components. FIG. 19 is a schematic illustration of a fusion protein according to an embodiment. The fusion protein includes an amino acid sequence of a WiChR variant according to an embodiment (“Opsin”), along with a fluorophore, an optional N-terminus signal sequence to improve membrane trafficking/expression, an optional Golgi trafficking signal sequence, an optional soma-targeting signal sequence, and an optional endoplasmic reticulum export signal sequence.

### Compositions

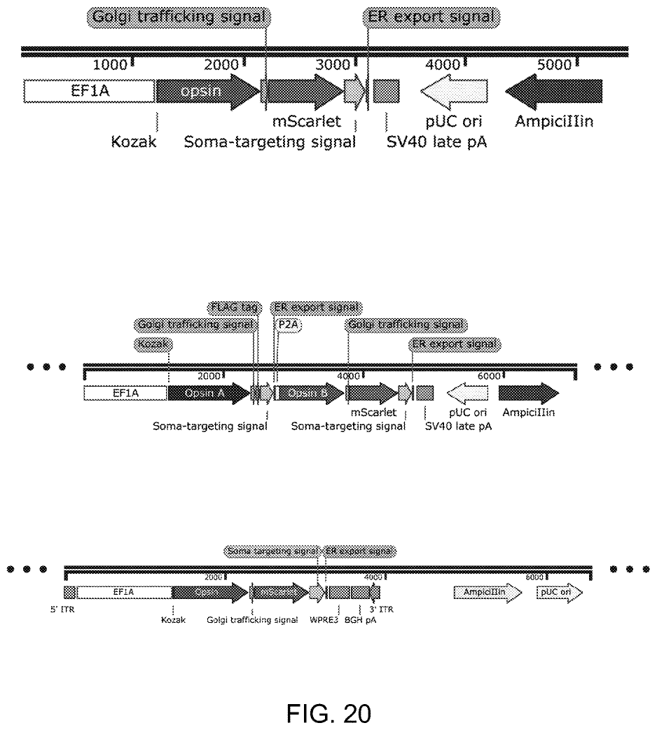

Several embodiments are directed to compositions that are useful in the delivery of WiChR variants according to embodiments, such as in methods of treatment. For example, WiChR variants according to embodiments can be packaged into adeno-associated viruses for delivery to a patient in gene therapy methods. FIG. 20 is a schematic illustration of a genetic construct payload for inclusion in an adeno-associated virus in a composition according to an embodiment. In the illustrated example, a nucleic acid encoding a WiChR variant according to an embodiment (“Opsin”) is under the control of the EF1A promoter, but any suitable promoter can be used, such as a promoter selected to target expression, to achieve conditional expression, or to target specific cell types (e.g. hSyn to target neurons). Also in the illustrated example, the AAV vector includes 5′ and 3′ inverted terminal repeat sequences, which are crucial to its packaging within an AAV capsid. It can also include a WPRE or WPRE3 sequence to improve transgene expression. A poly(A) signal (e.g., SV40 polyA or bovine growth hormone polyA) is incorporated to stabilize the mRNA transcript. AAV constructs according to embodiments can include additional modifications to provide or enhance compatibility with genetic systems for conditional expression, such as incorporating a double-floxed inverted open reading frame for use with Cre recombinase and analogous modifications for other recombinase systems such as Flp recombinase, Dre recombinase, etc. AAV constructs according to embodiments can include an alternate fluorophore or no fluorophore at all. AAV constructs according to embodiments can include additional or different antibiotic resistance cassettes, like Kanamycin.

The choice of AAV capsid is critical for targeted and efficient delivery. It is noted, though, that any of the various AAV serotypes may be employed, including AAV2, AAV8, AAV9, or other engineered capsids. A skilled artisan can select an appropriate AAV serotype based on any desired or required tropism, transduction efficiency, or reduced immunogenicity for retinal cells. In certain embodiments, the serotype is chosen to maximize infection of specific cell types in the retina, such as retinal ganglion cells, bipolar cells, etc. It is also noted that other delivery vectors, such as mRNA, CRISPR, Lentivirus, lipid nanoparticles, etc., can be used in compositions according to embodiments.

In compositions according to embodiments, AAV particles are suspended in a pharmaceutically acceptable carrier or buffer (e.g., PBS with magnesium and/or a surfactant). The viral titers can range from about 10\hat{ }10 to 10\hat{ }13 genome copies per mL, or higher as appropriate. The formulation can include stabilizers to maintain infectivity and reduce aggregate formation.

### Methods

WiChR variants according to embodiments are useful as excitatory optogenetic actuators of neural activity; they enable ‘write-in’ of information to the nervous system via exogenous light stimulation. This has therapeutic applications in diseases where neural activity is diminished or disrupted. Accordingly, several embodiments are directed to methods of treatment that include delivering a WiChR variant according to an embodiment to a patient in need thereof.

For example, several embodiments are directed to methods of restoring vision in a patient, such as a human being, having a form of blindness that involves degeneration of photoreceptors, such as retinitis pigmentosa, Leber congenital amaurosis, age-related macular degeneration, stargardt's disease, and other inherited or acquired degenerative diseases.

In these methods, excitatory optogenetic actuators comprising one or more WiChR variants according to one or more embodiments is introduced into remaining cells in the retina (such as bipolar cells or retinal ganglion cells) via gene therapy in order to restore photosensitivity. The gene therapy can be administered by various means. For example, in some embodiments, the AAV solution is delivered into the vitreous humor, allowing diffusion and transduction of the inner retina (e.g., ganglion or bipolar cells). In other embodiments, a small bleb is created between the retinal pigment epithelium (RPE) and the photoreceptor layer, ensuring direct transduction of bipolar or remaining photoreceptor cells. Suitable alternate approaches to introducing a WiChR variant according to an embodiment into remaining cells in the retina can be used.

A therapeutically effective dose for use in methods according to embodiments may vary depending on the AAV serotype, promoter, and patient condition. Typically, a single-eye dosage ranges from about 10\hat{ }10 to 10\hat{ }12 vg per eye. Bilateral treatment can be staged (e.g., treating one eye first to assess safety) or simultaneous, depending on clinical protocols, using the same or different dosage for each eye. A single injection may be sufficient for long-term expression given the stability of AAV transduction, but multiple injections (or “boosters”) might be administered if expression wanes or if additional therapeutic benefit is required. In methods according to certain embodiments, re-administration can occur months or years after initial dosing, subject to immunological considerations.

Adjunctive therapies, such as corticosteroids or other immune-modulating drugs, may be administered perioperatively to reduce inflammation or immune response to viral capsid proteins. Also, co-expression of agents that support retinal cell health may further enhance functional recovery.

Post-injection, retinal cells transduced by the AAV construct synthesize and express the WiChR fusion proteins, which are subsequently trafficked to their cellular membranes. These cells then become responsive to light stimuli.

Because certain WiChR variants respond robustly at very low irradiance levels (e.g., on the order of 0.1 μW/mm\hat{ }2), ordinary indoor or outdoor lighting may be sufficient to restore visual function. This is advantageous because patients are spared the burden of wearing goggles, headsets, or other specialized hardware. This can reduce overall treatment complexity, improve user comfort and compliance, and reduce costs associated with device manufacture, calibration, and maintenance. Furthermore, relying on ambient light preserves natural visual experiences and does not require users to learn new methods of visually scanning their environment with custom apparatus. This can accelerate adaptation and improve real-world functional outcomes (e.g., walking unassisted, recognizing faces, reading signs). Also, the elimination of the need for bulky headgear or bright projectors can improve patients' social interactions and day-to-day activities. There is no need to charge or calibrate external devices, which can increase adherence and overall satisfaction.

Many existing optogenetic therapies rely on extremely bright or focused illumination devices (e.g., goggles with built-in LED arrays) to compensate for lower opsin photosensitivity. These devices often require specialized optics, power sources, and heat management solutions. Even with these devices, the increased sensitivity of WiChR variants reduces power requirements, enabling a reduction in both heat and phototoxicity risk and the use of more Comfortable Light Levels, allowing patients to use devices at safer intensities without glare or discomfort.

Certain patients with extremely low residual retinal function or advanced disease may benefit from a moderate-intensity system to guarantee consistent light delivery, especially during nighttime or in dimly lit environments. Also, high-brightness devices can be programmed for interactive vision therapy sessions, helping patients train or optimize their newly restored photoreception.

Off-the-shelf VR/AR headsets often produce display luminances of 100-300 cd/m\hat{ }2 (or more), which can now be enough to trigger robust photocurrents in high-sensitivity WiChR-expressing cells. These systems allow dynamic, real-time modulation of visual scenes, which can be used for interactive rehabilitation, such as with software-driven training to improve visual acuity or field detection. Also, these devices can provide augmented cues, such as by providing overlaying guiding signals or contrasting edges to compensate for partial vision loss.

The Widespread availability of consumer headsets may reduce costs and logistical barriers. This enables patients to practice or perform rehabilitative exercises in a home environment. Many existing consumer VR/AR headsets have been designed with comfort and appearance in mind, and benefit from well-developed and stable software support. Repurposing these products for optogenetic stimulation would allow patients to enjoy these benefits, which are more difficult to achieve for specialized medical devices.

Certain patients may benefit from implantable devices that deliver either photostimulation directly to opsin-expressing cells or provide electrical stimulation as a complementary method of activating the retina. For photostimulation, an implant could allow for a light source to be placed in extremely close apposition to an opsin-expressing retinal cell. In combination with the high sensitivity of engineered WiChR variants, this could potentially greatly reduce the power requirements for photostimulation.

Optogenetic vision restoration via engineered WiChR variants could also be useful as a combination therapy in tandem with an electrically stimulating retinal implant. Current approaches to electrical stimulation of the retina use devices that cover only a small fraction of the retina (usually near the fovea), which means they can only restore visual input for a small portion of the visual field. Such a device could be complemented via optogenetic stimulation of the surrounding retina in order to allow for restoration of peripheral vision as well.

Standard tests (e.g., ETDRS charts, perimetry) can be used in patient follow-up to quantify improvements in vision under various lighting conditions (dark, low light, normal indoor light). Optical Coherence Tomography (OCT) can be performed to detect or track structural changes in retinal layers and confirms stable transgene expression over time. Electrophysiological assessments, such as electroretinography (ERG) and visually evoked potentials (VEP), can document functional improvements in retinal signaling and cortical visual processing.

Adverse event monitoring can also be performed, including monitoring for inflammation, such as signs of uveitis, vitritis, or choroiditis that might indicate immune response, and monitoring for infection or toxicity, such as monitoring for vector-related toxicity or off-target gene expression.

AAV-mediated gene expression can persist for years. Regular follow-up helps determine if re-administration is necessary. Patient-reported outcomes on quality of life parameters, such as ability to navigate indoors/outdoors, reading ability, and facial recognition, can be assessed to measure real-world benefits.

### NUMBERED STATEMENTS

1. A nucleic acid comprising the nucleotide sequence set forth in SEQ ID NO: 3.
2. A nucleic acid comprising the nucleotide sequence set forth in SEQ ID NO: 5.
3. A nucleic acid comprising the nucleotide sequence set forth in SEQ ID NO: 7.
4. A nucleic acid comprising the nucleotide sequence set forth in SEQ ID NO: 9.
5. A nucleic acid comprising the nucleotide sequence set forth in SEQ ID NO: 11.
6. A nucleic acid comprising the nucleotide sequence set forth in SEQ ID NO: 13.
7. A nucleic acid comprising the nucleotide sequence set forth in SEQ ID NO: 15.
8. A nucleic acid comprising the nucleotide sequence set forth in SEQ ID NO: 17.
9. A nucleic acid comprising the nucleotide sequence set forth in SEQ ID NO: 19.
10. A nucleic acid comprising the nucleotide sequence set forth in SEQ ID NO: 21.
11. A nucleic acid comprising the nucleotide sequence set forth in SEQ ID NO: 23.
12. A nucleic acid comprising the nucleotide sequence set forth in SEQ ID NO: 25.
13. A nucleic acid comprising the nucleotide sequence set forth in SEQ ID NO: 27.
14. A nucleic acid comprising the nucleotide sequence set forth in SEQ ID NO: 29.
15. A nucleic acid comprising the nucleotide sequence set forth in SEQ ID NO: 31.
16. A nucleic acid comprising the nucleotide sequence set forth in SEQ ID NO: 33.
17. A nucleic acid comprising the nucleotide sequence set forth in SEQ ID NO: 35.
18. A nucleic acid comprising the nucleotide sequence set forth in SEQ ID NO: 37.
19. A nucleic acid comprising the nucleotide sequence set forth in SEQ ID NO: 39.
20. A nucleic acid comprising the nucleotide sequence set forth in SEQ ID NO: 41.
21. A nucleic acid comprising the nucleotide sequence set forth in SEQ ID NO: 43.
22. A nucleic acid comprising the nucleotide sequence set forth in SEQ ID NO: 45.
23. A nucleic acid comprising the nucleotide sequence set forth in SEQ ID NO: 47.
24. A nucleic acid comprising the nucleotide sequence set forth in SEQ ID NO: 49.
25. A nucleic acid comprising the nucleotide sequence set forth in SEQ ID NO: 51.
26. A nucleic acid comprising the nucleotide sequence set forth in SEQ ID NO: 53.
27. A nucleic acid comprising the nucleotide sequence set forth in SEQ ID NO: 55.
28. A nucleic acid comprising the nucleotide sequence set forth in SEQ ID NO: 57.
29. A nucleic acid comprising the nucleotide sequence set forth in SEQ ID NO: 59.
30. A nucleic acid comprising the nucleotide sequence set forth in SEQ ID NO: 61.
31. A nucleic acid comprising the nucleotide sequence set forth in SEQ ID NO: 63.
32. A nucleic acid comprising the nucleotide sequence set forth in SEQ ID NO: 65.
33. A nucleic acid comprising the nucleotide sequence set forth in SEQ ID NO: 67.
34. A nucleic acid comprising the nucleotide sequence set forth in SEQ ID NO: 69.
35. A nucleic acid comprising the nucleotide sequence set forth in SEQ ID NO: 71.
36. A nucleic acid comprising the nucleotide sequence set forth in SEQ ID NO: 73.
37. A nucleic acid comprising the nucleotide sequence set forth in SEQ ID NO: 75.
38. A nucleic acid comprising the nucleotide sequence set forth in SEQ ID NO: 77.
39. A nucleic acid comprising the nucleotide sequence set forth in SEQ ID NO: 79.
40. A nucleic acid comprising the nucleotide sequence set forth in SEQ ID NO: 81.
41. A nucleic acid comprising the nucleotide sequence set forth in SEQ ID NO: 83.
42. A nucleic acid comprising the nucleotide sequence set forth in SEQ ID NO: 85.
43. A nucleic acid comprising the nucleotide sequence set forth in SEQ ID NO: 87.
44. A nucleic acid comprising the nucleotide sequence set forth in SEQ ID NO: 89.
45. A nucleic acid comprising the nucleotide sequence set forth in SEQ ID NO: 91.
46. A nucleic acid comprising the nucleotide sequence set forth in SEQ ID NO: 93.
47. A nucleic acid comprising the nucleotide sequence set forth in SEQ ID NO: 95.
48. A nucleic acid comprising the nucleotide sequence set forth in SEQ ID NO: 97.
49. A nucleic acid comprising the nucleotide sequence set forth in SEQ ID NO: 99.
50. A nucleic acid comprising the nucleotide sequence set forth in SEQ ID NO: 101.
51. A nucleic acid comprising the nucleotide sequence set forth in SEQ ID NO: 103.
52. A nucleic acid comprising the nucleotide sequence set forth in SEQ ID NO: 105.
53. A nucleic acid comprising the nucleotide sequence set forth in SEQ ID NO: 107.
54. A nucleic acid comprising the nucleotide sequence set forth in SEQ ID NO: 109.
55. A nucleic acid comprising the nucleotide sequence set forth in SEQ ID NO: 111.
56. A nucleic acid comprising the nucleotide sequence set forth in SEQ ID NO: 113.
57. A nucleic acid comprising the nucleotide sequence set forth in SEQ ID NO: 115.
58. A nucleic acid comprising the nucleotide sequence set forth in SEQ ID NO: 117.
59. A nucleic acid comprising the nucleotide sequence set forth in SEQ ID NO: 119.
60. A nucleic acid comprising the nucleotide sequence set forth in SEQ ID NO: 121.
61. A nucleic acid comprising the nucleotide sequence set forth in SEQ ID NO: 123.
62. A nucleic acid comprising the nucleotide sequence set forth in SEQ ID NO: 125.
63. A nucleic acid comprising the nucleotide sequence set forth in SEQ ID NO: 127.
64. A nucleic acid comprising the nucleotide sequence set forth in SEQ ID NO: 129.
65. A nucleic acid having at least 70%, 75%, 80%, 85%, 90%, 91%, 92%, 93%, 94%, 95%, 96%, 97%, 98%, or 99% sequence identity to a nucleotide sequence selected from SEQ ID NOs: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129.
66. A nucleic acid encoding a WiChR variant, wherein said nucleic acid comprises a nucleotide sequence encoding an amino acid sequence having at least 70%, 75%, 80%, 85%, 90%, 91%, 92%, 93%, 94%, 95%, 96%, 97%, 98%, or 99% sequence identity to an amino acid sequence selected from SEQ ID NOs: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130.
67. A nucleic acid comprising one or more point mutations selected from the point mutations described in SEQ ID NOs: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129 in comparison to the wild type nucleotide sequence presented in SEQ ID NO: 1.
68. A genetic construct comprising the nucleic acid of any one of numbered statements 1-64.
69. A genetic construct comprising a first nucleic acid according to any one of numbered statements 1-64 and a second nucleic acid according to any one of numbered statements 1-64.
70. The genetic construct of numbered statement 69, wherein the first nucleic acid and the second nucleic acid are the same.
71. The genetic construct of numbered statement 69, wherein the first nucleic acid and the second nucleic acid are different.
72. A host cell comprising the genetic construct of any of numbered statements 68-71.
73. A method of producing a WiChR variant, comprising expressing in a host cell a nucleic acid according to any of numbered statements 1-64.
74. A composition comprising a genetic construct according to any of numbered statements 68-71.
75. A composition comprising a genetic construct according to any of numbered statements 68-71 packaged in a vector.
76. The composition according to numbered statement 75, wherein the vector comprises a gene therapy vector.
77. The composition according to numbered statement 35, wherein the vector comprises an adeno-associated virus.
78. A protein comprising the amino acid sequence set forth in SEQ ID NO: 4.
79. A protein comprising the amino acid sequence set forth in SEQ ID NO: 6.
80. A protein comprising the amino acid sequence set forth in SEQ ID NO: 8.
81. A protein comprising the amino acid sequence set forth in SEQ ID NO: 10.
82. A protein comprising the amino acid sequence set forth in SEQ ID NO: 12.
83. A protein comprising the amino acid sequence set forth in SEQ ID NO: 14.
84. A protein comprising the amino acid sequence set forth in SEQ ID NO: 16.
85. A protein comprising the amino acid sequence set forth in SEQ ID NO: 18.
86. A protein comprising the amino acid sequence set forth in SEQ ID NO: 20.
87. A protein comprising the amino acid sequence set forth in SEQ ID NO: 22.
88. A protein comprising the amino acid sequence set forth in SEQ ID NO: 24.
89. A protein comprising the amino acid sequence set forth in SEQ ID NO: 26.
90. A protein comprising the amino acid sequence set forth in SEQ ID NO: 28.
91. A protein comprising the amino acid sequence set forth in SEQ ID NO: 30.
92. A protein comprising the amino acid sequence set forth in SEQ ID NO: 32.
93. A protein comprising the amino acid sequence set forth in SEQ ID NO: 34.
94. A protein comprising the amino acid sequence set forth in SEQ ID NO: 36.
95. A protein comprising the amino acid sequence set forth in SEQ ID NO: 38.
96. A protein comprising the amino acid sequence set forth in SEQ ID NO: 40.
97. A protein comprising the amino acid sequence set forth in SEQ ID NO: 42.
98. A protein comprising the amino acid sequence set forth in SEQ ID NO: 44.
99. A protein comprising the amino acid sequence set forth in SEQ ID NO: 46.
100. A protein comprising the amino acid sequence set forth in SEQ ID NO: 48.
101. A protein comprising the amino acid sequence set forth in SEQ ID NO: 50.
102. A protein comprising the amino acid sequence set forth in SEQ ID NO: 52.
103. A protein comprising the amino acid sequence set forth in SEQ ID NO: 54.
104. A protein comprising the amino acid sequence set forth in SEQ ID NO: 56.
105. A protein comprising the amino acid sequence set forth in SEQ ID NO: 58.
106. A protein comprising the amino acid sequence set forth in SEQ ID NO: 60.
107. A protein comprising the amino acid sequence set forth in SEQ ID NO: 62.
108. A protein comprising the amino acid sequence set forth in SEQ ID NO: 64.
109. A protein comprising the amino acid sequence set forth in SEQ ID NO: 66.
110. A protein comprising the amino acid sequence set forth in SEQ ID NO: 68.
111. A protein comprising the amino acid sequence set forth in SEQ ID NO: 70.
112. A protein comprising the amino acid sequence set forth in SEQ ID NO: 72.
113. A protein comprising the amino acid sequence set forth in SEQ ID NO: 74.
114. A protein comprising the amino acid sequence set forth in SEQ ID NO: 76.
115. A protein comprising the amino acid sequence set forth in SEQ ID NO: 78.
116. A protein comprising the amino acid sequence set forth in SEQ ID NO: 80.
117. A protein comprising the amino acid sequence set forth in SEQ ID NO: 82.
118. A protein comprising the amino acid sequence set forth in SEQ ID NO: 84.
119. A protein comprising the amino acid sequence set forth in SEQ ID NO: 86.
120. A protein comprising the amino acid sequence set forth in SEQ ID NO: 88.
121. A protein comprising the amino acid sequence set forth in SEQ ID NO: 90.
122. A protein comprising the amino acid sequence set forth in SEQ ID NO: 92.
123. A protein comprising the amino acid sequence set forth in SEQ ID NO: 94.
124. A protein comprising the amino acid sequence set forth in SEQ ID NO: 96.
125. A protein comprising the amino acid sequence set forth in SEQ ID NO: 98.
126. A protein comprising the amino acid sequence set forth in SEQ ID NO: 100.
127. A protein comprising the amino acid sequence set forth in SEQ ID NO: 102.
128. A protein comprising the amino acid sequence set forth in SEQ ID NO: 104.
129. A protein comprising the amino acid sequence set forth in SEQ ID NO: 106.
130. A protein comprising the amino acid sequence set forth in SEQ ID NO: 108.
131. A protein comprising the amino acid sequence set forth in SEQ ID NO: 110.
132. A protein comprising the amino acid sequence set forth in SEQ ID NO: 112.
133. A protein comprising the amino acid sequence set forth in SEQ ID NO: 114.
134. A protein comprising the amino acid sequence set forth in SEQ ID NO: 116.
135. A protein comprising the amino acid sequence set forth in SEQ ID NO: 118.
136. A protein comprising the amino acid sequence set forth in SEQ ID NO: 120.
137. A protein comprising the amino acid sequence set forth in SEQ ID NO: 122.
138. A protein comprising the amino acid sequence set forth in SEQ ID NO: 124.
139. A protein comprising the amino acid sequence set forth in SEQ ID NO: 126.
140. A protein comprising the amino acid sequence set forth in SEQ ID NO: 128.
141. A protein comprising the amino acid sequence set forth in SEQ ID NO: 130.
142. A protein having at least 70%, 75%, 80%, 85%, 90%, 91%, 92%, 93%, 94%, 95%, 96%, 97%, 98%, or 99% sequence identity to any of SEQ ID NOs: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130.
143. A method of treatment, comprising delivering a protein according to any of numbered statements 78-141 to a patient in need thereof.
144. A method of restoring vision in a patient having a form of blindness that involves degeneration of photoreceptors, the method comprising introducing a protein according to any of numbered statements 78-141 into remaining cells in the retina of said patient via gene therapy to restore photosensitivity.
145. A nucleic acid comprising a nucleotide sequence set forth in any odd-numbered SEQ ID NO. between SEQ ID NOS. 3 and 350.
146. A nucleic acid having at least 70%, 75%, 80%, 85%, 90%, 91%, 92%, 93%, 94%, 95%, 96%, 97%, 98%, or 99% sequence identity to a nucleotide sequence set forth in any odd-numbered SEQ ID NO. between SEQ ID NOS. 3 and 350.
147. A protein comprising an amino acid sequence set forth in any odd-numbered SEQ ID NO. between SEQ ID NOS. 3 and 350.
148. A protein having at least 70%, 75%, 80%, 85%, 90%, 91%, 92%, 93%, 94%, 95%, 96%, 97%, 98%, or 99% sequence identity to an amino acid sequence set forth in any even-numbered SEQ ID NO. between SEQ ID NOS. 3 and 350.

### REFERENCES

Berndt, A., Schoenenberger, P., Mattis, J., Tye, K. M., Deisseroth, K., Hegemann, P., & Oertner, T. G. (2011). High-efficiency channelrhodopsins for fast neuronal stimulation at low light levels. Proc. Natl. Acad. Sci. U.S.A, 108 (18), 7595-7600.
Govorunova, E. G., Gou, Y., Sineshchekov, O. A., Li, H., Lu, X., Wang, Y., Brown, L. S., St-Pierre, F., Xue, M., & Spudich, J. L. (2022). Kalium channelrhodopsins are natural light-gated potassium channels that mediate optogenetic inhibition. Nat. Neurosci., 25 (7), 967-974.
Govorunova, E. G., Sineshchekov, O. A., Brown, L. S., Bondar, A.-N., & Spudich, J. L. (2022). Structural Foundations of Potassium Selectivity in Channelrhodopsins. MBio, 13 (6), e0303922.
Mattis, J., Tye, K. M., Ferenczi, E. A., Ramakrishnan, C., O'Shea, D. J., Prakash, R., Gunaydin, L. A., Hyun, M., Fenno, L. E., Gradinaru, V., Yizhar, O., & Deisseroth, K. (2011). Principles for applying optogenetic tools derived from direct comparative analysis of microbial opsins. Nat. Methods, 9 (2), 159-172.
Pan, Z.-H., Ganjawala, T. H., Lu, Q., Ivanova, E., & Zhang, Z. (2014). ChR2 mutants at L132 and T159 with improved operational light sensitivity for vision restoration. PLOS One, 9 (6), e98924.
Shepard, B. D., Natarajan, N., Protzko, R. J., Acres, O. W., & Pluznick, J. L. (2013). A cleavable N-terminal signal peptide promotes widespread olfactory receptor surface expression in HEK293T cells. PLOS One, 8 (7), e68758.
Tajima, S., Kim, Y. S., Fukuda, M., Jo, Y., Wang, P. Y., Paggi, J. M., Inoue, M., Byrne, E. F. X., Kishi, K. E., Nakamura, S., Ramakrishnan, C., Takaramoto, S., Nagata, T., Konno, M., Sugiura, M., Katayama, K., Matsui, T. E., Yamashita, K., Kim, S., . . . . Kato, H. E. (2023). Structural basis for ion selectivity in potassium-selective channelrhodopsins. Cell, 186 (20), 4325-4344.e26.
Takaramoto, S., Fainsod, S., Nagata, T., Rozenberg, A., Béjà, O., & Inoue, K. (2024). HulaCCR1, a pump-like cation channelrhodopsin discovered in a lake microbiome. J. Mol. Biol., 436 (23), 168844.
Tose, A., Nava, A., McGrath, S., Mardinly, A., Naka, A., (2025). WAChRs are excitatory opsins sensitive to indoor lighting. BioRxiv, https://doi.org/10.1101/2025.09.12.675947.
Vierock, J., Peter, E., Grimm, C., Rozenberg, A., Chen, I.-W., Tillert, L., Castro Scalise, A. G., Casini, M., Augustin, S., Tanese, D., Forget, B. C., Peyronnet, R., Schneider-Warme, F., Emiliani, V., Béjà, O., & Hegemann, P. (2022). WiChR, a highly potassium-selective channelrhodopsin for low-light one- and two-photon inhibition of excitable cells. Sci Adv, 8 (49), eadd7729.
Watanabe, Y., Sugano, E., Tabata, K., Hatakeyama, A., Sakajiri, T., Fukuda, T., Ozaki, T., Suzuki, T., Sayama, T., & Tomita, H. (2021). Development of an optogenetic gene sensitive to daylight and its implications in vision restoration. NPJ Regen Med, 6 (1), 64.
Yan, B., Viswanathan, S., Brodie, S. E., Deng, W.-T., Coleman, K. E., Hauswirth, W. W., & Nirenberg, S. (2023). A clinically viable approach to restoring visual function using optogenetic gene therapy. Mol. Ther. Methods Clin. Dev., 29, 406-417.
Zerche, M., Hunniford, V., Alekseev, A., El May, F., Vavakou, A., Siegenthaler, D., Hüser, M. A., Kiehn, S. M., Garrido-Charles, A., Alvanos, T., Witzke, I., Trenholm, S., Mace, E., Kusch, K., Bruegmann, T., Wolf, B. J., Mager, T., & Moser, T. (2023). Efficient and sustained optogenetic control of nervous and cardiac systems. BioRxiv, 2023.11.17.567544.
Zhang, M., Shan, Y., & Pei, D. (2022). Cryo-EM structures of kalium channelrhodopsins KCRs. BioRxiv, 2022.11.09.515798.

The foregoing detailed description refers to example WiChR variants, nucleic acids that encode a WiChR variant, genetic constructs that include a nucleic acid encoding a WiChR variant, compositions that include a nucleic acid encoding a WiChR variant, and methods of treatment. The description and the appended drawings illustrating the described WiChR variants, nucleic acids, genetic constructs, compositions, and methods of treatment are intended only to provide examples and not to limit the scope of the claims in any manner.

What is claimed is:

1. A nucleic acid comprising a nucleotide sequence having at least about 95% sequence identity to the sequence set forth in SEQ ID NO: 3.

2. The nucleic acid of claim 1, wherein the nucleic acid encodes an amino acid sequence comprising an F240A substitution relative to the amino acid sequence set forth in SEQ ID NO. 2.

3. The nucleic acid of claim 1, wherein the nucleic acid encodes an amino acid sequence comprising an L130F substitution relative to the amino acid sequence set forth in SEQ ID NO. 2.

4. The nucleic acid of claim 1, wherein the nucleic acid encodes an amino acid sequence comprising an S122L substitution relative to the amino acid sequence set forth in SEQ ID NO. 2.

5. The nucleic acid of claim 1, wherein the nucleic acid encodes an amino acid sequence comprising an F211W substitution relative to the amino acid sequence set forth in SEQ ID NO. 2.

6. The nucleic acid of claim 1, wherein the nucleic acid encodes an amino acid sequence comprising D109N, S122L, and R196Q substitutions relative to the amino acid sequence set forth in SEQ ID NO. 2.

7. A genetic construct comprising the nucleic acid of claim 2.

8. The genetic construct of claim 7, further comprising a promoter operably linked to the nucleic acid.

9. A mammalian cell comprising the genetic construct of claim 8.

10. A composition comprising a genetic construct according to claim 8 packaged in a vector.

11. The composition according to claim 10, wherein the vector comprises a gene therapy vector.

12. The composition according to claim 11, wherein the vector comprises an adeno-associated virus.

13. The nucleic acid of claim 2, wherein the nucleotide sequence has at least about 97% sequence identity to the sequence set forth in SEQ ID NO: 3.

14. A protein comprising an amino acid sequence having at least about 95% sequence identity to the sequence set forth in SEQ ID NO: 4.

15. The protein of claim 14, wherein the amino acid sequence comprises an F240A substitution relative to the amino acid sequence set forth in SEQ ID NO. 2.

16. The protein of claim 14, wherein the amino acid sequence further comprises an L130F substitution relative to the amino acid sequence set forth in SEQ ID NO. 2.

17. The protein of claim 14, wherein the amino acid sequence further comprises an S122L substitution relative to the amino acid sequence set forth in SEQ ID NO. 2.

18. The protein of claim 14, wherein the amino acid sequence further comprises an F211W substitution relative to the amino acid sequence set forth in SEQ ID NO. 2.

19. The protein of claim 14, wherein the amino acid sequence further comprises D109N, S122L, and R196Q substitutions relative to the amino acid sequence set forth in SEQ ID NO. 2.

20. A method of restoring vision in a patient having a form of blindness that involves degeneration of photoreceptors, the method comprising introducing a protein according to claim 15 into remaining cells in the retina of said patient via gene therapy to restore photosensitivity.


SEQUENCE LISTING (350 sequences)

SEQ ID NO: 1 - DNA, 933 bp, Wobblia lunata
  source 1..933: mol_type = other DNA
atgagcgcctgccccaccccccccaccgtggtggcccccaccgaccagagccccaaccccaacggcggcaccaccagctgcgcccaggccgcccccagcccccagatgagcatgaactacaccctgctggccaccatcgacggcttcagcgccgtgatcctgctgggcagcggcgtggtgttctggtactacagctggcgcggcaccggcttcggcgcccacccccaccccaagcgccgccccctggagctgaacaccctgagcgccgccatcgccttcctgaccggcctgatgtacgtggccaagaccagcgacttcgcccgcgacagcaccagcgagttcagcctgaaccgctactggtacagcgactacatcgtgacctgccccctgatcgtgatggacctgtgcttcaccgtggacgtgccctacaagctggccttcaccggcagcaccgccgtgctgctggtgatcgccctggccagcttcatcgtgcgcagccccgagaagtacatctacttcgccatcggctgcaccctgttcagcatcttcttctacttcttcttcaacgaggtgcgcaagcgcatccgctacgtgcccgactgcgccaagaaggacctggagcgcgccatgttcatcttcttcggcttctggcccatgttccccgtgctgtggctgctgggcttccacaccaccggcgtgatcagcgaggaggccttcttcatcatccacgccttcctggacatcacctgcaagaccatcttcggcgtgttcatgctgcgctgccgcctgcacatcgaggactacgcctgggacgagctgaagcgccagcgccaggagatcgacgaggagaaccagaagaccctgaccgccgccggcgccgacggcgccaccgtgatgatgcccgtggaggaggacgacccctacgccagcgga

SEQ ID NO: 2 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFFIIHAFLDITCKTIFGVFMLRCRLHIEDYAWDELKRQRQEIDEENQKTLTAAGADGATVMMPVEEDDPYASG

SEQ ID NO: 3 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacattgtgacctgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattttcttttacttctttttcaatgaggtgagaaagaggattaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 4 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 5 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaaggccgacttggagagggccatgttcatcttcttcggcttctggccctgcttccccgttctgtggctgttaggcttccacaccaccggcgtgatcagcgaggaggcattcgccattattcacgccctgctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 6 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKADLERAMFIFFGFWPCFPVLWLLGFHTTGVISEEAFAIIHALLDITCKTIFGVFMLRCRLHIEDYAWDELKRQRQEIDEENQKTLTAAGADGATVMMPVEEDDPYASG

SEQ ID NO: 7 - DNA, 933 bp, Wobblia lunata
  source 1..933: mol_type = other DNA
atgagcgcctgccccacaccccctaccgtggtggcccccaccgatcaaagccccaaccctaacggcggcaccaccagctgcgcccaggccgccccttctccccagatgagcatgaattacaccctgctggccaccatcgacggcttcagcgccgtgatcctgctgggaagcggcgtggtgttctggtactacagctggaggggcaccggcttcggcgcccacccccatcccaagagaagacccctggagctgaacaccctgagcgccgccatcgccttcttgaccggcctgatgtacgtggccaagaccagcgacttcgccagggacagcaccagcgagttcagcctgaacaggtactggtacagcgactacatcgtggtgtgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggcttccacaccaccggcgtgatcagcgaggaggcctttgccatcatccacgccttcctggacatcacctgcaagaccatcttcggcgtgttcatgctgaggtgcagactgcacatcgaggactacgcctgggacgagctgaagaggcagagacaggagatcgacgaggagaaccagaagaccttgaccgccgctggcgccgacggcgccaccgtgatgatgcccgtggaggaagacgacccctacgctagcggc

SEQ ID NO: 8 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVVCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 9 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacattgtgacctgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattttcttttacttctttttcaatgaggtgagaaagaggattaggtacgtgcccgactgcgccaagaaggacctggagagggccatgttcatcttcttcggcttctggcccctgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 10 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 11 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacattgtggtgtgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattttcttttacttctttttcaatgaggtgagaaagaggattaggtacgtgcccgacgccgccaagaaggacctggagagggccatgttcatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 12 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVVCPLIVMDLCFTVDVPYKLAFTGSTAVLLVIALASFIVRSPEKYIYFAIGCTLFSIFFYFFFNEVRKRIRYVPDAAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 13 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggattaggtacgtgcccgacgccgccaagaaggacctggagagggccatgttcatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcggc

SEQ ID NO: 14 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDAAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 15 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttagccggcctgatgtacgtggccaagaccagcgactttgctagggacagcaccagcgagtttagcctgaacaggtactggtacagcgactacattgtgacctgccctctggtggtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattttcttttacttctttttcaatgaggtgagaaagaggattaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcggc

SEQ ID NO: 16 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYSDYIVTCPLVVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 17 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctatttcgctatcggctgcaccctgtttagcattttcttctacttctttttcaacgaggtgagaaagaggatcaggtacgtgcccgactgcgccaagaaggacctggagagggccatgttcatctacttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 18 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIYFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 19 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgtacatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 20 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMYIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 21 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacttcgagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 22 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDFERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 23 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcaacttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 24 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAINFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 25 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctactacgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 26 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYYAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 27 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattatgttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 28 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIMFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 29 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacctggactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 30 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 31 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattatgttctacttctttttcaacgaggtgagaaagaggatcaggtacgtgcccgactgcgccaagaaggacctggagagggccatgtacatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 32 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIMFYFFFNEVRKRIRYVPDCAKKDLERAMYIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 33 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatatacttcgctatcggctgcaccctgtttagcattttcttctacttctttttcaacgaggtgagaaagaggatcaggtacgtgcccgactgcgccaagaaggacctggagagggccatgtacatctacttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 34 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMYIYFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 35 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctatttcgctatcggctgcaccctgtttagcattttcttctacttctttttcaacgaggtgagaaagaggatcaggtacgtgcccgactgcgccaagaaggacctggagagggccatgttcatctacttcggcttctggcccctgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 36 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIYFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 37 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttagccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctatttcgctatcggctgcaccctgtttagcattttcttctacttctttttcaacgaggtgagaaagaggatcaggtacgtgcccgactgcgccaagaaggacctggagagggccatgttcatctacttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 38 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIYFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 39 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtatatctatttcgctatcggctgcaccctgtttagcattttcctgtacttctttttcaacgaggtgagaaagaggatcaggtacgtgcccgactgcgccaagaaggacctggagagggccatgttcatctacttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 40 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFLYFFFNEVRKRIRYVPDCAKKDLERAMFIYFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 41 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcagcggatttagcgccgtgattctgcttggaagcggcgtggtgttctggtactactcttggaggggaaccggctttggagcccacccccatcccaagagaagacccctggaactgaataccctgtctgccgccatcaacttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 42 - protein, 311 aa, Wobblia lunata
MSACPTPPTVVAPTDQSPNPNGGTTSCAQAAPSPQMSMNYTLLATISGFSAVILLGSGVVFWYYSWRGTGFGAHPHPKRRPLELNTLSAAINFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 43 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacattgtgacctgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattttcttttacttctttttcaatgaggtgagaaagaggattaggtacgtgcccgactgcgccaagaaggacctggagagggccatgttcttattcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 44 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFLFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 45 - DNA, 933 bp, Wobblia lunata
  source 1..933: mol_type = other DNA
atgagcgcctgccccacaccccctaccgtggtggcccccaccgatcaaagccccaaccctaacggcggcaccaccagctgcgcccaggccgccccttctccccagatgagcatgaattacaccctgctggccaccatcgacggcttcagcgccgtgatcctgctgggaagcggcgtggtgttctggtactacagctggaggggcaccggcttcggcgcccacccccatcccaagagaagacccctggagctgaacaccctgagcgccgccatcgccttcttgaccggcctgatgtacgtggccaagaccagcgacttcgccagggacagcaccagcgagttcagcctgaacaggtactggtacagcgactacatcgtgacctgccccctgatcgtgatggacctgtgcttcaccgtggacgtgccctacaagctggccttcaccggcagcaccgccgtgctgctggtgatcggcctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggcttccacaccaccggcgtgatcagcgaggaggcctttgccatcatccacgccttcctggacatcacctgcaagaccatcttcggcgtgttcatgctgaggtgcagactgcacatcgaggactacgcctgggacgagctgaagaggcagagacaggagatcgacgaggagaaccagaagaccttgaccgccgctggcgccgacggcgccaccgtgatgatgcccgtggaggaagacgacccctacgctagcggc

SEQ ID NO: 46 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIGLASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 47 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgctgagatactggtacagcgactacattgtgacctgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattttcttttacttctttttcaatgaggtgagaaagaggattaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 48 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLLRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 49 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacattgtgacctgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattttcttttacttctttatcaatgaggtgagaaagaggattaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcttagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 50 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFINEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 51 - DNA, 1056 bp, Wobblia lunata
  source 1..1056: mol_type = other DNA
atgagaaggaaactggcgctgacatctctgtgcggcatgcccggcaaactggctgcggcgtgtatcctggctacagtggtggtcagcttcgccgccccatctatccccagcatccaaagcgtgatgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacattgtgacctgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattttcttttacttctttttcaatgaggtgagaaagaggattaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 52 - protein, 352 aa, Wobblia lunata
MRRKLALTSLCGMPGKLAAACILATVVVSFAAPSIPSIQSVMSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 53 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacgccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 54 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHATGVISEEAFAIIHAFLDITCKTIFGVFMLRCRLHIEDYAWDELKRQRQEIDEENQKTLTAAGADGATVMMPVEEDDPYASG

SEQ ID NO: 55 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttttggcccatgttccccgtgctgtggctgctgggctttcacaccaccggcgtgatcagcgaggaggcctttgctatcattcacgccttcctagacattacctgcaagaccattttcggcttcttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 56 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGFFMLRCRLHIEDYAWDELKRQRQEIDEENQKTLTAAGADGATVMMPVEEDDPYASG

SEQ ID NO: 57 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagctggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 58 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLELAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 59 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccgtgttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 60 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTVFGVFMLRCRLHIEDYAWDELKRQRQEIDEENQKTLTAAGADGATVMMPVEEDDPYASG

SEQ ID NO: 61 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctgagaagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 62 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLRRAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 63 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactggccatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 64 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLAIEDYAWDELKRQRQEIDEENQKTLTAAGADGATVMMPVEEDDPYASG

SEQ ID NO: 65 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttctactacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 66 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFYYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 67 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacgccgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 68 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYADYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 69 - DNA, 933 bp, Wobblia lunata
  source 1..933: mol_type = other DNA
atgagcgcctgtcctacaccccctaccgtggtggcccccacagatcaaagccccaatcctaacggaggcaccacctctgccgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcggc

SEQ ID NO: 70 - protein, 311 aa, Wobblia lunata
MSACPTPPTVVAPTDQSPNPNGGTTSAAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 71 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgccgccgatgagctgaaaaggcagagacaggagatcgacgaggagaatcagaaaaccttgaccgccgctggcgccgatggcgccaccgtgatgatgcccgttgaagaagacgacccctacgctagcggc

SEQ ID NO: 72 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAADELKRQRQEIDEENQKTLTAAGADGATVMMPVEEDDPYASG

SEQ ID NO: 73 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtatatctatttcgctatcggctgcaccctgtttagcattttcctgtacttctttttcaacgaggtgagaaagaggatcaggtacgtgcccgactgcgccaagaaggacctggagagggccatgttcatctacttcggcttctggcccctgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 74 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFLYFFFNEVRKRIRYVPDCAKKDLERAMFIYFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 75 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgaaacagcaccagcgagtttagcctgaacaggtactggtacctggactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 76 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARNSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 77 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtggtggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 78 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVVDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 79 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcagaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 80 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFQTTGVISEEAFAIIHAFLDITCKTIFGVFMLRCRLHIEDYAWDELKRQRQEIDEENQKTLTAAGADGATVMMPVEEDDPYASG

SEQ ID NO: 81 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttatcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 82 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFIIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 83 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacctggactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgctgatcttcttcggcttctggcccatgttccccgttctgtggctgttaggcttccacaccaccggcgtgatcagcgaggaggcattcgccattattcacgccttcctggacatcacatgcaaaaccatcttcggcgtgttcctgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 84 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMLIFFGFWPMFPVLWLLGFHTTGVISEEAFAIIHAFLDITCKTIFGVFLLRCRLHIEDYAWDELKRQRQEIDEENQKTLTAAGADGATVMMPVEEDDPYASG

SEQ ID NO: 85 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttagacgagacagcaccagcgagtttagcctgaacaggtactggtacgccgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggccagatcagcgaggaggcattcgccattattcacgccttcctggacatcagctgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 86 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFRRDSTSEFSLNRYWYADYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGQISEEAFAIIHAFLDISCKTIFGVFMLRCRLHIEDYAWDELKRQRQEIDEENQKTLTAAGADGATVMMPVEEDDPYASG

SEQ ID NO: 87 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacgccgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcctgtacttctttttcaatgaggtgagaaagagaatcaggtacgtgcccgactgcgccaagaaggacttggagaggctgatgttcatcttcgccggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 88 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYADYIVTCPLIVMDLCFTVDVPYKLAFTGSTAVLLVIALASFIVRSPEKYIYFAIGCTLFSIFLYFFFNEVRKRIRYVPDCAKKDLERLMFIFAGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 89 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttctgagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacctggactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccaccttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 90 - protein, 311 aa, Wobblia lunata
MSACPTPPTVVAPTDQSPNPNGGTTSCAQAAPSPQMSMNYTLLATIDGFSAVILLLSGVVFWYYSWRGTGFGAHPHPKRRPLELNTLSAAIAFLTGLMYVAKTSDFARDSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERATFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 91 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaaggccgacttggagagggccatgttcatcttcttcggcttctggcccatgttccccgttctgttcctgttaggcttccacaccaccggcgtgatcagcgaggaggcattcgccattattcacgccctgctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 92 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKADLERAMFIFFGFWPMFPVLFLLGFHTTGVISEEAFAIIHALLDITCKTIFGVFMLRCRLHIEDYAWDELKRQRQEIDEENQKTLTAAGADGATVMMPVEEDDPYASG

SEQ ID NO: 93 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtggccctgcttctgagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccgtgttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 94 - protein, 311 aa, Wobblia lunata
MSACPTPPTVVAPTDQSPNPNGGTTSCAQAAPSPQMSMNYTLLATIDGFSAVALLLSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTVFGVFMLRCRLHIEDYAWDELKRQRQEIDEENQKTLTAAGADGATVMMPVEEDDPYASG

SEQ ID NO: 95 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcatcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggccctgcttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 96 - protein, 311 aa, Wobblia lunata
MSACPTPPTVVAPTDQSPNPNGGTTSCAQAAPSPQMSMNYTLLATIDGISAVILLGSGVVFWYYSWRGTGFGAHPHPKRRPLELNTLSAAIAFLAGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPCFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 97 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagtgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacctggactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggccctgcttccccgttctgtggctgttaggcttcagcaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 98 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPCFPVLWLLGFSTTGVISEEAFAIIHAFLDITCKTIFGVFMLRCRLHIEDYAWDELKRQRQEIDEENQKTLTAAGADGATVMMPVEEDDPYASG

SEQ ID NO: 99 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtggccctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttagctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcagccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 100 - protein, 311 aa, Wobblia lunata
MSACPTPPTVVAPTDQSPNPNGGTTSCAQAAPSPQMSMNYTLLATIDGFSAVALLGSGVVFWYYSWRGTGFGAHPHPKRRPLELNTLSAAIAFLTGLMYVAKTSDFARDSTSEFSLNRYWYSDYIVSCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGSHTTGVISEEAFAIIHAFLDITCKTIFGVFMLRCRLHIEDYAWDELKRQRQEIDEENQKTLTAAGADGATVMMPVEEDDPYASG

SEQ ID NO: 101 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgaccgccgccatcgccttcttaaccggcctgatgtacgtggccaagaccagcgactttgctaggaacagcaccagcgagtttagcctgaacaggtactggtacctggactacattgtgacctgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattttcttttacttctttttcaatgaggtgagaaagaggattaggtacgtgcccgactgcgccaagaaggacctggagagggccatgttcatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 102 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLTAAIAFLTGLMYVAKTSDFARNSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 103 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtatctgtacttagactacatcgttacctgtcccctgctggtcatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcctgtacttctttttcaatgaggtgagaaagagaatcaggtacgtgcccgactgcgccaagaaggacttggagagggccatgttcatcttcttcggcttctggcccctgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 104 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYLYLDYIVTCPLLVMDLCFTVDVPYKLAFTGSTAVLLVIALASFIVRSPEKYIYFAIGCTLFSIFLYFFFNEVRKRIRYVPDCAKKDLERAMFIFFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 105 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtatctgtacttagactacatcgttacctgtcccctgctggtcatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcctgtacttctttttcaatgaggtgagaaagagaatcaggtacgtgcccgactgcgccaagaaggacttggagagggccatgttcatcttcttcggcttctggcccgccttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 106 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYLYLDYIVTCPLLVMDLCFTVDVPYKLAFTGSTAVLLVIALASFIVRSPEKYIYFAIGCTLFSIFLYFFFNEVRKRIRYVPDCAKKDLERAMFIFFGFWPAFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 107 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccgccttaaccggcctgatgtacgtggccaagaccagcgactttgctagggacagcaccagcgagtttagcctgaacaggtactggtacagcgactacattgtgacctgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattatgttttacttctttttcaatgaggtgagaaagaggattaggtacgtgcccgactgcgccaagaaggacctggagagggccatgtacatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 108 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAALTGLMYVAKTSDFARDSTSEFSLNRYWYSDYIVTCPLIVMDLCFTVDVPYKLAFTGSTAVLLVIALASFIVRSPEKYIYFAIGCTLFSIMFYFFFNEVRKRIRYVPDCAKKDLERAMYIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 109 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctaggaacagcaccagcgagtttagcctgaacaggtactggtacctggactacattgtgacctgccctctgattgtgatggacttatgtttcaccgttgacgttccctacaagctggcattcaccggaagcaccgccgtgctgctggtgattgccttagccagctttattgtgaggagccccgagaagtatgcctactttgcaattggctgcacactgtttagcattttcttttacttctttttcaatgaggtgagaaagaggattaggtacgtgcccgactgcgccaagaaggacctggagagggccatgttcatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 110 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARNSTSEFSLNRYWYLDYIVTCPLIVMDLCFTVDVPYKLAFTGSTAVLLVIALASFIVRSPEKYAYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 111 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatctacttcggcttctggcccctgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 112 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIYFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 113 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtactggtacctggactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcctgtacttctttttcaatgaggtgagaaagagaatcaggtacgtgcccgactgcgccaagaaggacttggagagggccatgttcatctacttcggcttctggcccctgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 114 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYLDYIVTCPLIVMDLCFTVDVPYKLAFTGSTAVLLVIALASFIVRSPEKYIYFAIGCTLFSIFLYFFFNEVRKRIRYVPDCAKKDLERAMFIYFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 115 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcagcggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtacttatacagcgactacatcgttacctgtcccctggtggtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 116 - protein, 311 aa, Wobblia lunata
MSACPTPPTVVAPTDQSPNPNGGTTSCAQAAPSPQMSMNYTLLATISGFSAVILLGSGVVFWYYSWRGTGFGAHPHPKRRPLELNTLSAAIAFLAGLMYVAKTSDFARDSTSEFSLNRYLYSDYIVTCPLVVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 117 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcagcggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtactggtacagcgactacatcgttacctgtcccctggtggtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 118 - protein, 311 aa, Wobblia lunata
MSACPTPPTVVAPTDQSPNPNGGTTSCAQAAPSPQMSMNYTLLATISGFSAVILLGSGVVFWYYSWRGTGFGAHPHPKRRPLELNTLSAAIAFLAGLMYVAKTSDFARDSTSEFSLNRYWYSDYIVTCPLVVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 119 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtcattctgcttggaagcgccgtggtgttctggtactactcttggaggggaaccggctttggagcccacccccatcccaagagaagacccctggaactgaataccctgtctgccgccatcgccttcttaaccggcctgatgtacgtggccaagaccagcgactttgctaggaacagcaccagcgagtttagcctgaacaggtactggtacctggactacattgtgacctgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattttcttttacttctttttcaatgaggtgagaaagaggattaggtacgtgcccgactgcgccaagaaggacctggagagggccatgttcatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 120 - protein, 311 aa, Wobblia lunata
MSACPTPPTVVAPTDQSPNPNGGTTSCAQAAPSPQMSMNYTLLATIDGFSAVILLGSAVVFWYYSWRGTGFGAHPHPKRRPLELNTLSAAIAFLTGLMYVAKTSDFARNSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 121 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcagcggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtactggtacctggactacatcgttacctgtcccctggtggtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 122 - protein, 311 aa, Wobblia lunata
MSACPTPPTVVAPTDQSPNPNGGTTSCAQAAPSPQMSMNYTLLATISGFSAVILLGSGVVFWYYSWRGTGFGAHPHPKRRPLELNTLSAAIAFLAGLMYVAKTSDFARDSTSEFSLNRYWYLDYIVTCPLVVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 123 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactacgcctggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacattgtgacctgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattatgttttacttctttttcaatgaggtgagaaagaggattaggtacgtgcccgactgcgccaagaaggacctggagagggccatgttcatcttcttcggcttctggcccctgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 124 - protein, 311 aa, Wobblia lunata
MSACPTPPTVVAPTDQSPNPNGGTTSCAQAAPSPQMSMNYTLLATIDGFSAVILLGSGVVFWYYAWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIMFYFFFNEVRKRIRYVPDCAKKDLERAMFIFFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 125 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtatctgtacttagactacatcgttacctgtcccctgctggtcatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcctgtacttctttttcaatgaggtgagaaagagaatcaggtacgtgcccgactgcgccaagaaggacttggagagggccatgttcatctacttcggcttctggcccctgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 126 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYLYLDYIVTCPLLVMDLCFTVDVPYKLAFTGSTAVLLVIALASFIVRSPEKYIYFAIGCTLFSIFLYFFFNEVRKRIRYVPDCAKKDLERAMFIYFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 127 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtactggtacagcgactacatcgttgcctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatctacttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 128 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYSDYIVACPLIVMDLCFTVDVPYKLAFTGSTAVLLVIALASFIVRSPEKYIYFAIGCTLFSIFFYFFFNEVRKRIRYVPDCAKKDLERAMFIYFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 129 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcttcaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 130 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSFNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 131 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcaacttcttaaccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttatcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 132 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAINFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFINEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 133 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgccggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 134 - protein, 311 aa, Wobblia lunata
MSACPTPPTVVAPTDQSPNPNGGTTSCAQAAPSPQMSMNYTLLATIAGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 135 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgccggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcttcattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 136 - protein, 311 aa, Wobblia lunata
MSACPTPPTVVAPTDQSPNPNGGTTSCAQAAPSPQMSMNYTLLATIAGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFFIIHAFLDITCKTIFGVFMLRCRLHIEDYAWDELKRQRQEIDEENQKTLTAAGADGATVMMPVEEDDPYASG

SEQ ID NO: 137 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatctgcggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgtaccccgttctgtggctgttaggcttccacaccaccggcgtgatcagcgaggaggcattcttcattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 138 - protein, 311 aa, Wobblia lunata
MSACPTPPTVVAPTDQSPNPNGGTTSCAQAAPSPQMSMNYTLLATICGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMYPVLWLLGFHTTGVISEEAFFIIHAFLDITCKTIFGVFMLRCRLHIEDYAWDELKRQRQEIDEENQKTLTAAGADGATVMMPVEEDDPYASG

SEQ ID NO: 139 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatctgcggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacattgtgacctgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattttcttttacttctttttcaatgaggtgagaaagaggattaggtacgtgcccgactgcgccaagaaggacctggagagggccatgttcatcttcttcggcttctggcccatgttccccgtgctgtggctgctgggctttcacaccaccggcgtgatcagcgaggaggcctttttcatcatccacgccttcctggacatcacctgcaagaccatcttcggcgtgttcatgctgaggtgcagactgcacatcgaggactacgcctgggatgagctgaaaaggcagagacaggagatcgacgaggagaaccagaagaccttgaccgccgctggcgccgacggcgccaccgtgatgatgcccgtggaggaagacgacccctacgctagcggc

SEQ ID NO: 140 - protein, 311 aa, Wobblia lunata
MSACPTPPTVVAPTDQSPNPNGGTTSCAQAAPSPQMSMNYTLLATICGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFFIIHAFLDITCKTIFGVFMLRCRLHIEDYAWDELKRQRQEIDEENQKTLTAAGADGATVMMPVEEDDPYASG

SEQ ID NO: 141 - DNA, 933 bp, Wobblia lunata; identical to SEQ ID NO: 75
  source 1..933: mol_type = other DNA

SEQ ID NO: 142 - protein, 311 aa, Wobblia lunata; identical to SEQ ID NO: 76

SEQ ID NO: 143 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgaaacagcaccagcgagtttagcctgaacaggtactggtacctggactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccctgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 144 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARNSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 145 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctaggaacagcaccagcgagtttagcctgaacaggtactggtacctggactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatacagtacgtgcccgactgcgccaagaaggacctggagagggccatgttcatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 146 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARNSTSEFSLNRYWYLDYIVTCPLIVMDLCFTVDVPYKLAFTGSTAVLLVIALASFIVRSPEKYIYFAIGCTLFSIFFYFFFNEVRKRIQYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 147 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctaggaacagcaccagcgagtttagcctgaacaggtactggtacctggactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccagaaaggacctggagagggccatgttcatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 148 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARNSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCARKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 149 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctaggaacagcaccagcgagtttagcctgaacaggtactggtacctggactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaaggccgacctggagagggccatgttcatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 150 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARNSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKADLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 151 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctaggaacagcaccagcgagtttagcctgaacaggtactggtacctggactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgagctgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 152 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARNSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLSCRLHIEDYAWDELKRQRQEIDEENQKTLTAAGADGATVMMPVEEDDPYASG

SEQ ID NO: 153 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctaggaacagcaccagcgagtttagcctgaacaggtactggtacctggactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaagcagagacaggagatcgacgaggagaatcagaaaaccttgaccgccgctggcgccgatggcgccaccgtgatgatgcccgttgaagaagacgacccctacgctagcgga

SEQ ID NO: 154 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARNSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKKQRQEIDEENQKTLTAAGADGATVMMPVEEDDPYASG

SEQ ID NO: 155 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctaggaacagcaccagcgagtttagcctgaacaggtactggtacctggactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatacagtacgtgcccgactgcgccaaggccgacctggagagggccatgttcatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 156 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARNSTSEFSLNRYWYLDYIVTCPLIVMDLCFTVDVPYKLAFTGSTAVLLVIALASFIVRSPEKYIYFAIGCTLFSIFFYFFFNEVRKRIQYVPDCAKADLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 157 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctaggaacagcaccagcgagtttagcctgaacaggtactggtacctggactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagcaacaggagatcgacgaggagaatcagaaaaccttgaccgccgctggcgccgatggcgccaccgtgatgatgcccgttgaagaagacgacccctacgctagcgga

SEQ ID NO: 158 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARNSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQQQEIDEENQKTLTAAGADGATVMMPVEEDDPYASG

SEQ ID NO: 159 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgagtacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 160 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSEYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 161 - DNA, 933 bp, Wobblia lunata; identical to SEQ ID NO: 31
  source 1..933: mol_type = other DNA

SEQ ID NO: 162 - protein, 311 aa, Wobblia lunata; identical to SEQ ID NO: 32

SEQ ID NO: 163 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtatatctacttcgctatcggctgcaccctgtttagcattttcctgtacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 164 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFLYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 165 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgtggatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 166 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMWIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 167 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttctacggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 168 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFYGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 169 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgtaccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 170 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMYPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 171 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgtaccccgttctgtggctgttaggcttccacaccaccggcgtgatcagcgaggaggcattcagcattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 172 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMYPVLWLLGFHTTGVISEEAFSIIHAFLDITCKTIFGVFMLRCRLHIEDYAWDELKRQRQEIDEENQKTLTAAGADGATVMMPVEEDDPYASG

SEQ ID NO: 173 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcgccgacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 174 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFADITCKTIFGVFMLRCRLHIEDYAWDELKRQRQEIDEENQKTLTAAGADGATVMMPVEEDDPYASG

SEQ ID NO: 175 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcacagtggacgtgccctacaagctggccttcaccgtgagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 176 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTVSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 177 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctggtggtgatggacctgtgcttcaccgtggacgtgccctacaagctggccttcaccggcagcaccgccgtgctgctggtgatcgccctcgccagcttcatcgtgaggagccccgagaagtacatctatttcgctatcggctgcaccctgtttagcattttcttctacttctttttcaacgaggtgagaaagaggatcaggtacgtgcccgactgcgccaagaaggacctggagagggccatgttcatctacttcggcttctggcccatgtaccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 178 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLVVMDLCFTVDVPYKLAFTGSTAVLLVIALASFIVRSPEKYIYFAIGCTLFSIFFYFFFNEVRKRIRYVPDCAKKDLERAMFIYFGFWPMYPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 179 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacattgtgacctgccctctgattgtgatggacttatgtttcaccgttgacgttccctacaagctggcattcaccggaagcaccgccgtgctgctggtgattgccttagccagctttctggtgaggagccccgagaagtatatttactttgcaattggctgcacactgtttagcattatgttttacttctttttcaatgaggtgagaaagaggattaggtacgtgcccgactgcgccaagaaggacctggagagggccatgttcatcttcttcggcttctggcccctgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 180 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFLVRSPEKYIYFAIGCTLFSIMFYFFFNEVRKRIRYVPDCAKKDLERAMFIFFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 181 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagcgggccaggtacgtgcccgactgcgccaagaaggacttggagagggccatgttcatcttcttcggcttctggccctgcttccccgttctgtggctgttaggcttccacaccaccggcgtgatcagcgaggaggcattcgccattattcacgccctgctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 182 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRARYVPDCAKKDLERAMFIFFGFWPCFPVLWLLGFHTTGVISEEAFAIIHALLDITCKTIFGVFMLRCRLHIEDYAWDELKRQRQEIDEENQKTLTAAGADGATVMMPVEEDDPYASG

SEQ ID NO: 183 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccagaaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 184 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCARKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 185 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaaggccgacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 186 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKADLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 187 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttacctgtcccttcatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 188 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPFIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 189 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccttcatcgtcatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgtaccccgtgctgtggctgctgggctttcacaccaccggcgtgatcagcgaggaggcctttttcatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 190 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPFIVMDLCFTVDVPYKLAFTGSTAVLLVIALASFIVRSPEKYIYFAIGCTLFSIFFYFFFNEVRKRIRYVPDCAKKDLERAMFIFFGFWPMYPVLWLLGFHTTGVISEEAFFIIHAFLDITCKTIFGVFMLRCRLHIEDYAWDELKRQRQEIDEENQKTLTAAGADGATVMMPVEEDDPYASG

SEQ ID NO: 191 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccttcatcgtcatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttttcatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 192 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPFIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFFIIHAFLDITCKTIFGVFMLRCRLHIEDYAWDELKRQRQEIDEENQKTLTAAGADGATVMMPVEEDDPYASG

SEQ ID NO: 193 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttacctgtcccctgatcgtgatggacttctgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 194 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDFCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 195 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatactgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctggccgtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 196 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLAVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 197 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtggtgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 198 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVVWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 199 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggccctgctaccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 200 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPCYPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 201 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccttcttccccgttctgtggctgttaggcttcgacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 202 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPFFPVLWLLGFDTTGVISEEAFAIIHAFLDITCKTIFGVFMLRCRLHIEDYAWDELKRQRQEIDEENQKTLTAAGADGATVMMPVEEDDPYASG

SEQ ID NO: 203 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacattgtgacctgccctctgattgtgatggacttatgtttcaccgttgacgttccctacaagctggcattcaccggaagcaccgccgtgctgctggtgattgccttagccagctttattgtggacagccccgagaagtatatttactttgcaattggctgcacactgtttagcattatgttttacttctttttcaatgaggtgagaaagaggattaggtacgtgcccgactgcgccaagaaggacctggagagggccatgtacatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 204 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVDSPEKYIYFAIGCTLFSIMFYFFFNEVRKRIRYVPDCAKKDLERAMYIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 205 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatacagtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 206 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIQYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 207 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacgccgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaaggccgacttggagagggccatgttcatcttcttcggcttctggcccatgttccccgttctgtggctgttaggcttccacaccaccggcgtgatcagcgaggaggcattcgccattattcacgccttcctggacatcacatgcaaaaccgtgttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 208 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYADYIVTCPLIVMDLCFTVDVPYKLAFTGSTAVLLVIALASFIVRSPEKYIYFAIGCTLFSIFFYFFFNEVRKRIRYVPDCAKADLERAMFIFFGFWPMFPVLWLLGFHTTGVISEEAFAIIHAFLDITCKTVFGVFMLRCRLHIEDYAWDELKRQRQEIDEENQKTLTAAGADGATVMMPVEEDDPYASG

SEQ ID NO: 209 - DNA, 933 bp, Wobblia lunata; identical to SEQ ID NO: 29
  source 1..933: mol_type = other DNA

SEQ ID NO: 210 - protein, 311 aa, Wobblia lunata; identical to SEQ ID NO: 30

SEQ ID NO: 211 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacctggactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattatgttttacttctttttcaatgaggtgagaaagagaatcaggtacgtgcccgactgcgccaagaaggacttggagagggccatgttcatcttcttcggcttctggcccctgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 212 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYLDYIVTCPLIVMDLCFTVDVPYKLAFTGSTAVLLVIALASFIVRSPEKYIYFAIGCTLFSIMFYFFFNEVRKRIRYVPDCAKKDLERAMFIFFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 213 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacctggactacatcgtgacatgccccttcatcgtcatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttttcatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 214 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYLDYIVTCPFIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFFIIHAFLDITCKTIFGVFMLRCRLHIEDYAWDELKRQRQEIDEENQKTLTAAGADGATVMMPVEEDDPYASG

SEQ ID NO: 215 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacctggactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttttcatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 216 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFFIIHAFLDITCKTIFGVFMLRCRLHIEDYAWDELKRQRQEIDEENQKTLTAAGADGATVMMPVEEDDPYASG

SEQ ID NO: 217 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttagccggcctgatgtacgtggccaagaccagcgactttgctagggacagcaccagcgagtttagcctgaacaggtactggtacagcgactacattgtgacctgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattttcttttacttctttttcaatgaggtgagaaagaggattaggtacgtgcccgactgcgccaagaaggacctggagagggccatgttcatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 218 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 219 - DNA, 933 bp, Wobblia lunata; identical to SEQ ID NO: 113
  source 1..933: mol_type = other DNA

SEQ ID NO: 220 - protein, 311 aa, Wobblia lunata; identical to SEQ ID NO: 114

SEQ ID NO: 221 - DNA, 933 bp, Wobblia lunata; identical to SEQ ID NO: 125
  source 1..933: mol_type = other DNA

SEQ ID NO: 222 - protein, 311 aa, Wobblia lunata; identical to SEQ ID NO: 126

SEQ ID NO: 223 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtactggtacctggactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcctgtacttctttttcaatgaggtgagaaagagaatcaggtacgtgcccgactgcgccaagaaggacttggagagggccatgttcatctacttcggcttctggccctgcttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 224 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYLDYIVTCPLIVMDLCFTVDVPYKLAFTGSTAVLLVIALASFIVRSPEKYIYFAIGCTLFSIFLYFFFNEVRKRIRYVPDCAKKDLERAMFIYFGFWPCFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 225 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttagccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctatttcgctatcggctgcaccctgtttagcattttcttctacttctttttcaacgaggtgagaaagaggatcaggtacgtgcccgactgcgccaagaaggacctggagagggccatgttcatctacttcggcttctggcccatgttccccgtgctgtggctgctgggctttcacaccaccggcgtgatcagcgaggaggcctttttcatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 226 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIYFGFWPMFPVLWLLGFHTTGVISEEAFFIIHAFLDITCKTIFGVFMLRCRLHIEDYAWDELKRQRQEIDEENQKTLTAAGADGATVMMPVEEDDPYASG

SEQ ID NO: 227 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttagccggcctgatgtacgtggccaagaccagcgactttgctagggacagcaccagcgagtttagcctgaacaggtactggtacagcgactacatcgtgacatgccccttcatcgtcatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttttcatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 228 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYSDYIVTCPFIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFFIIHAFLDITCKTIFGVFMLRCRLHIEDYAWDELKRQRQEIDEENQKTLTAAGADGATVMMPVEEDDPYASG

SEQ ID NO: 229 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttagccggcctgatgtacgtggccaagaccagcgactttgctagggacagcaccagcgagtttagcctgaacaggtactggtacagcgactacatcgtgacatgccccttcgtggtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttttcatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 230 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYSDYIVTCPFVVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFFIIHAFLDITCKTIFGVFMLRCRLHIEDYAWDELKRQRQEIDEENQKTLTAAGADGATVMMPVEEDDPYASG

SEQ ID NO: 231 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttagccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctactttgctatcggctgcaccctgtttagcattttcttctacttctttttcaacgaggtgagaaagaggatcaggtacgtgcccgactgcgccaagaaggacctggagagggccatgttcatctacttcggcttctggcccatgtaccccgtgctgtggctgctgggctttcacaccaccggcgtgatcagcgaggaggcctttttcatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 232 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIYFGFWPMYPVLWLLGFHTTGVISEEAFFIIHAFLDITCKTIFGVFMLRCRLHIEDYAWDELKRQRQEIDEENQKTLTAAGADGATVMMPVEEDDPYASG

SEQ ID NO: 233 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttatgcggcctgatgtacgtggccaagaccagcgactttgctagggacagcaccagcgagtttagcctgaacaggtactggtacctggactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttttcatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 234 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLCGLMYVAKTSDFARDSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFFIIHAFLDITCKTIFGVFMLRCRLHIEDYAWDELKRQRQEIDEENQKTLTAAGADGATVMMPVEEDDPYASG

SEQ ID NO: 235 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacattgtgagctgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattttcttttacttctttttcaatgaggtgagaaagaggattaggtacgtgcccgactgcgccaagaaggacctggagagggccatgttcatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 236 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVSCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 237 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacattgtggtgtgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattttcttttacttctttttcaatgaggtgagaaagaggattaggtacgtgcccgacgccgccaagaaggacctggagagggccatgttcatcttcttcggcttctggcccatgttccccgtgctgtggctgctgggctttcacaccaccggcgtgatcagcgaggaggcctttgccatcattcacgccttcctagacattacctgcaaggcgattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcggc

SEQ ID NO: 238 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVVCPLIVMDLCFTVDVPYKLAFTGSTAVLLVIALASFIVRSPEKYIYFAIGCTLFSIFFYFFFNEVRKRIRYVPDAAKKDLERAMFIFFGFWPMFPVLWLLGFHTTGVISEEAFAIIHAFLDITCKAIFGVFMLRCRLHIEDYAWDELKRQRQEIDEENQKTLTAAGADGATVMMPVEEDDPYASG

SEQ ID NO: 239 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttgtctgccccctgatcgtgatggacctgtgcttcaccgtggacgtgccttacaaactggcttttaccggcagcaccgctgttatgctggtgatcggcctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcttcattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 240 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVVCPLIVMDLCFTVDVPYKLAFTGSTAVMLVIGLASFIVRSPEKYIYFAIGCTLFSIFFYFFFNEVRKRIRYVPDCAKKDLERAMFIFFGFWPMFPVLWLLGFHTTGVISEEAFFIIHAFLDITCKTIFGVFMLRCRLHIEDYAWDELKRQRQEIDEENQKTLTAAGADGATVMMPVEEDDPYASG

SEQ ID NO: 241 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtcattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtggtgtgccccctgatcgtgatggacctgtgcttcaccgtggacgtgccctacaagctggccttcaccggcagcaccgccgtgatgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgacgccgccaagaaggacctggagagggccatgttcatcttcttcggcttctggcccatgttccccgtgctgtggctgctgggctttcacaccaccggcgtgatcagcgaggaggcctttttcatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 242 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVVCPLIVMDLCFTVDVPYKLAFTGSTAVMLVIALASFIVRSPEKYIYFAIGCTLFSIFFYFFFNEVRKRIRYVPDAAKKDLERAMFIFFGFWPMFPVLWLLGFHTTGVISEEAFFIIHAFLDITCKTIFGVFMLRCRLHIEDYAWDELKRQRQEIDEENQKTLTAAGADGATVMMPVEEDDPYASG

SEQ ID NO: 243 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtaccagtacctggactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccggcttccccgttctgtggctgttaggcttccacaccaccggcgtgatcagcgaggaggcattcgccattattcacgccttcctggacatcacatgcaaaaccgtgttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 244 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYQYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPGFPVLWLLGFHTTGVISEEAFAIIHAFLDITCKTVFGVFMLRCRLHIEDYAWDELKRQRQEIDEENQKTLTAAGADGATVMMPVEEDDPYASG

SEQ ID NO: 245 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtacacctacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 246 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYTYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 247 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactattacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccgccttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 248 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYYYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPAFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 249 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgttcctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 250 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLFLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 251 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccataatcgccttcttaaccggcctgatgtacgtggccaagaccagcgactttgctagggacagcaccagcgagtttagcctgaacaggtactggtacagcgactacattgtgacctgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattatgttttacttctttttcaatgaggtgagaaagaggattaggtacgtgcccgactgcgccaagaaggacctggagagggccatgttcatcttcttcggcttctggcccctgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 252 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAIIAFLTGLMYVAKTSDFARDSTSEFSLNRYWYSDYIVTCPLIVMDLCFTVDVPYKLAFTGSTAVLLVIALASFIVRSPEKYIYFAIGCTLFSIMFYFFFNEVRKRIRYVPDCAKKDLERAMFIFFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 253 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcaacttcttaaccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggccctgcttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 254 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAINFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPCFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 255 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgtgcgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatctacttcggcttctggcccctgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 256 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFVRDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIYFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 257 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagtgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgtgcgagacagcaccagcgagtttagcctgaacaggtactggtacctggactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggccctgcttccccgttctgtggctgttaggcttcagcaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 258 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFVRDSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPCFPVLWLLGFSTTGVISEEAFAIIHAFLDITCKTIFGVFMLRCRLHIEDYAWDELKRQRQEIDEENQKTLTAAGADGATVMMPVEEDDPYASG

SEQ ID NO: 259 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcacctccgagtttagcctgaacaggtactggtacagcgactacattgtgacctgccctctgattgtgatggacttatgtttcaccgttgacgttccctacaagctggcattcaccggaagcaccctggtgctgctggtgattgccttagccagctttattgtgaggagccccgagaagtatatttactttgcaattggctgcacactgtttagcattatgttttacttctttttcaatgaggtgagaaagaggattaggtacgtgcccgactgcgccaagaaggacctggagagggccatgtacatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 260 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTLVLLVIALASFIVRSPEKYIYFAIGCTLFSIMFYFFFNEVRKRIRYVPDCAKKDLERAMYIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 261 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacaacccccttaatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 262 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTTPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 263 - DNA, 933 bp, Wobblia lunata; identical to SEQ ID NO: 117
  source 1..933: mol_type = other DNA

SEQ ID NO: 264 - protein, 311 aa, Wobblia lunata; identical to SEQ ID NO: 118

SEQ ID NO: 265 - DNA, 933 bp, Wobblia lunata; identical to SEQ ID NO: 121
  source 1..933: mol_type = other DNA

SEQ ID NO: 266 - protein, 311 aa, Wobblia lunata; identical to SEQ ID NO: 122

SEQ ID NO: 267 - DNA, 933 bp, Wobblia lunata; identical to SEQ ID NO: 115
  source 1..933: mol_type = other DNA

SEQ ID NO: 268 - protein, 311 aa, Wobblia lunata; identical to SEQ ID NO: 116

SEQ ID NO: 269 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcagcggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtactggtacctggactacatcgttacctgtcccctggtggtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttatcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 270 - protein, 311 aa, Wobblia lunata
MSACPTPPTVVAPTDQSPNPNGGTTSCAQAAPSPQMSMNYTLLATISGFSAVILLGSGVVFWYYSWRGTGFGAHPHPKRRPLELNTLSAAIAFLAGLMYVAKTSDFARDSTSEFSLNRYWYLDYIVTCPLVVMDLCFTVDVPYKLAFTGSTAVLLVIALASFIVRSPEKYIYFAIGCTLFSIFFYFFINEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 271 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatctacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttctttgtgttctttttcaatgaggtgagaaagagaatcaggtacgtgcccgactgcgccaagaaggacttggagagggccatgttcatctacttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 272 - protein, 311 aa, Wobblia lunata
MSACPTPPTVVAPTDQSPNPNGGTTSCAQAAPSPQMSMNYTLLATIYGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFVFFFNEVRKRIRYVPDCAKKDLERAMFIYFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 273 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctaggaacagcaccagcgagtttagcctgaacaggtactggtacctggactacattgtgacctgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgctgagcattttcttttacttctttttcaatgaggtgagaaagaggattaggtacgtgcccgactgcgccaagaaggacctggagagggccatgttcatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 274 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARNSTSEFSLNRYWYLDYIVTCPLIVMDLCFTVDVPYKLAFTGSTAVLLVIALASFIVRSPEKYIYFAIGCTLLSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 275 - DNA, 933 bp, Wobblia lunata; identical to SEQ ID NO: 61
  source 1..933: mol_type = other DNA

SEQ ID NO: 276 - protein, 311 aa, Wobblia lunata; identical to SEQ ID NO: 62

SEQ ID NO: 277 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattatgttctacttctttttcaacgaggtgagaaagaggatcaggtacgtgcccgactgcgccaagaaggacctggagagggccatgttcatcttcttcggcttctggcccctgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 278 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIMFYFFFNEVRKRIRYVPDCAKKDLERAMFIFFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 279 - DNA, 933 bp, Wobblia lunata; identical to SEQ ID NO: 73
  source 1..933: mol_type = other DNA

SEQ ID NO: 280 - protein, 311 aa, Wobblia lunata; identical to SEQ ID NO: 74

SEQ ID NO: 281 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtatatctacttcgctatcggctgcaccctgtttagcattttcctgtacttctttttcaacgaggtgagaaagaggatcaggtacgtgcccgactgcgccaagaaggacctggagaggctgatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 282 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFLYFFFNEVRKRIRYVPDCAKKDLERLMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 283 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttctactacttctttttcaacgaggtgagaaagaggatcaggtacgtgcccgactgcgccaagaaggacctggagagggccatgttcatcttcttcggcttctggcccctgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 284 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFYYFFFNEVRKRIRYVPDCAKKDLERAMFIFFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 285 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttctactacttctttttcaacgaggtgagaaagaggatcaggtacgtgcccgactgcgccaagaaggacctggagagggccatgtacatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 286 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFYYFFFNEVRKRIRYVPDCAKKDLERAMYIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 287 - DNA, 933 bp, Wobblia lunata; identical to SEQ ID NO: 59
  source 1..933: mol_type = other DNA

SEQ ID NO: 288 - protein, 311 aa, Wobblia lunata; identical to SEQ ID NO: 60

SEQ ID NO: 289 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacatcgtgacatgccccctggtggtgatggacctgtgcttcaccgtggacgtgccctacaagctggccttcaccggcagcaccgccgtgctgctggtgatcgccctcgccagcttcatcgtgaggagccccgagaagtatatctacttcgctatcggctgcaccctgtttagcattttcctgtacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 290 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLVVMDLCFTVDVPYKLAFTGSTAVLLVIALASFIVRSPEKYIYFAIGCTLFSIFLYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 291 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcatcaccggcctgatgtacgtggccaaaacaagcgactttgcccgagacagcaccagcgagtttagcctgaacaggtactggtacagcgactacatcgttgtctgccccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacctgtttttcaatgaggtgagaaagagaatcaggtacgtgcccgacgccgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccgtgttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 292 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFITGLMYVAKTSDFARDSTSEFSLNRYWYSDYIVVCPLIVMDLCFTVDVPYKLAFTGSTAVLLVIALASFIVRSPEKYIYFAIGCTLFSIFFYLFFNEVRKRIRYVPDAAKKDLERAMFIFFGFWPMFPVLWLLGFHTTGVISEEAFAIIHAFLDITCKTVFGVFMLRCRLHIEDYAWDELKRQRQEIDEENQKTLTAAGADGATVMMPVEEDDPYASG

SEQ ID NO: 293 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacattgtgacctgccctctgattgtgatggacttatgtttcaccgttgacgttccctacaagctggcattcaccggaagcaccgccgtgctgatcgtgattgccttagccagctttattgtgaggagccccgagaagtatatttactttgcaattggctgcacactgtttagcattttcttttacttctttttcaatgaggtgagaaagaggattaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 294 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLIVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 295 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctggtggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 296 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLVVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 297 - DNA, 933 bp, Wobblia lunata; identical to SEQ ID NO: 101
  source 1..933: mol_type = other DNA

SEQ ID NO: 298 - protein, 311 aa, Wobblia lunata; identical to SEQ ID NO: 102

SEQ ID NO: 299 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacgccgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagaggctgatgttcatcttcgccggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 300 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYADYIVTCPLIVMDLCFTVDVPYKLAFTGSTAVLLVIALASFIVRSPEKYIYFAIGCTLFSIFFYFFFNEVRKRIRYVPDCAKKDLERLMFIFAGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 301 - DNA, 933 bp, Wobblia lunata; identical to SEQ ID NO: 87
  source 1..933: mol_type = other DNA

SEQ ID NO: 302 - protein, 311 aa, Wobblia lunata; identical to SEQ ID NO: 88

SEQ ID NO: 303 - DNA, 933 bp, Wobblia lunata; identical to SEQ ID NO: 67
  source 1..933: mol_type = other DNA

SEQ ID NO: 304 - protein, 311 aa, Wobblia lunata; identical to SEQ ID NO: 68

SEQ ID NO: 305 - DNA, 933 bp, Wobblia lunata; identical to SEQ ID NO: 97
  source 1..933: mol_type = other DNA

SEQ ID NO: 306 - protein, 311 aa, Wobblia lunata; identical to SEQ ID NO: 98

SEQ ID NO: 307 - DNA, 933 bp, Wobblia lunata; identical to SEQ ID NO: 81
  source 1..933: mol_type = other DNA

SEQ ID NO: 308 - protein, 311 aa, Wobblia lunata; identical to SEQ ID NO: 82

SEQ ID NO: 309 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggactgggcttcggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacagcgactacattgtgacctgccctctgattgtgatggacttatgtttcaccgttgacgttccctacaagctggcattcaccggaagcaccgccgtgctgctggtgattgccttagccagctttattgtgaggagccccgagaagtatatttactttgcaattggctgcacactgtttagcattatgttttacttctttttcaatgaggtgagaaagaggattaggtacgtgcccgactgcgccaagaaggacctggagagggccatgtacatcttcttcggcttctggcccatgttccccgtgctgtggctgctgggctttcacaccaccggcgtgatcagcgaggaggcctttgccatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 310 - protein, 311 aa, Wobblia lunata
MSACPTPPTVVAPTDQSPNPNGGTTSCAQAAPSPQMSMNYTLLATIDGFSAVILLGSGVVFWYYSWRGLGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIMFYFFFNEVRKRIRYVPDCAKKDLERAMYIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 311 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtactggtacctggactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcctgtacttctttttcaatgaggtgagaaagagaatcaggtacgtgcccgactgcgccaagaaggacttggagagggccatgttcatctacttcggcttctggcccctgttccccgttctgtggctgttaggcttccacaccaccggcgtgatcagcgaggaggcattcgccattattcacgccttcctggacatcacatgcaaaaccatcttcggcgtgttcatgctgaggtgtagactggccatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 312 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYLDYIVTCPLIVMDLCFTVDVPYKLAFTGSTAVLLVIALASFIVRSPEKYIYFAIGCTLFSIFLYFFFNEVRKRIRYVPDCAKKDLERAMFIYFGFWPLFPVLWLLGFHTTGVISEEAFAIIHAFLDITCKTIFGVFMLRCRLAIEDYAWDELKRQRQEIDEENQKTLTAAGADGATVMMPVEEDDPYASG

SEQ ID NO: 313 - DNA, 933 bp, Wobblia lunata; identical to SEQ ID NO: 105
  source 1..933: mol_type = other DNA

SEQ ID NO: 314 - protein, 311 aa, Wobblia lunata; identical to SEQ ID NO: 106

SEQ ID NO: 315 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtactggtacctggactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatctacttcggcttctggcccctgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 316 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIYFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 317 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtactggtacctggactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcctgtacttctttttcaatgaggtgagaaagagaatcaggtacgtgcccgactgcgccaaggccgacttggagagggccatgttcatctacttcggcttctggcccctgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 318 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYLDYIVTCPLIVMDLCFTVDVPYKLAFTGSTAVLLVIALASFIVRSPEKYIYFAIGCTLFSIFLYFFFNEVRKRIRYVPDCAKADLERAMFIYFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 319 - DNA, 933 bp, Wobblia lunata; identical to SEQ ID NO: 111
  source 1..933: mol_type = other DNA

SEQ ID NO: 320 - protein, 311 aa, Wobblia lunata; identical to SEQ ID NO: 112

SEQ ID NO: 321 - DNA, 933 bp, Wobblia lunata; identical to SEQ ID NO: 103
  source 1..933: mol_type = other DNA

SEQ ID NO: 322 - protein, 311 aa, Wobblia lunata; identical to SEQ ID NO: 104

SEQ ID NO: 323 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtacttatacagcgactacatcgttacctgtcccctggtggtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 324 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYLYSDYIVTCPLVVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 325 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtactggtacctggactacatcgttacctgtcccctggtggtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagaggctgatgttcatcttcttcggcttctggcccatgtaccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 326 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYLDYIVTCPLVVMDLCFTVDVPYKLAFTGSTAVLLVIALASFIVRSPEKYIYFAIGCTLFSIFFYFFFNEVRKRIRYVPDCAKKDLERLMFIFFGFWPMYPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 327 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaatcagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgactgcgccaagaaggacttggagagggccatgttcatctacttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 328 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKISDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIYFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 329 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtactggtacagcgactacatcgttacctgtcccctggtggtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcctgtacttctttttcaatgaggtgagaaagagaatcaggtacgtgcccgactgcgccaagaaggacttggagagggccatgttcatctacttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 330 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYSDYIVTCPLVVMDLCFTVDVPYKLAFTGSTAVLLVIALASFIVRSPEKYIYFAIGCTLFSIFLYFFFNEVRKRIRYVPDCAKKDLERAMFIYFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 331 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtactggtacagcgactacatcgttacctgtcccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcctgtacttctttttcaatgaggtgagaaagagaatcaggtacgtgcccgactgcgccaagaaggacttggagagggccatgttcatctacttcggcttctggcccctgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 332 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYSDYIVTCPLIVMDLCFTVDVPYKLAFTGSTAVLLVIALASFIVRSPEKYIYFAIGCTLFSIFLYFFFNEVRKRIRYVPDCAKKDLERAMFIYFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 333 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttagccggcctgatgtacgtggccaaaacaagcgactttgcccgagacagcaccagcgagtttagcctgaacaggtactggtacctggactacatcgttacctgtcccctggtggtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcctgtacttctttttcaatgaggtgagaaagagaatcaggtacgtgcccgactgcgccaagaaggacttggagagggccatgttcatctacttcggcttctggcccctgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 334 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLAGLMYVAKTSDFARDSTSEFSLNRYWYLDYIVTCPLVVMDLCFTVDVPYKLAFTGSTAVLLVIALASFIVRSPEKYIYFAIGCTLFSIFLYFFFNEVRKRIRYVPDCAKKDLERAMFIYFGFWPLFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 335 - DNA, 933 bp, Wobblia lunata; identical to SEQ ID NO: 127
  source 1..933: mol_type = other DNA

SEQ ID NO: 336 - protein, 311 aa, Wobblia lunata; identical to SEQ ID NO: 128

SEQ ID NO: 337 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttgtctgccccctgatcgtgatggacctgtgcttcaccgtggacgtgccttacaaactggcttttaccggcagcaccgctgttctgctggtgatcgccctggccagcttcattgtgagaagccccgagaaatacatttactttgccattggctgtaccctgtttagcattttcttttacttctttttcaatgaggtgagaaagagaatcaggtacgtgcccgacgccgccaagaaggacttcgagagggccatgtacatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 338 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVVCPLIVMDLCFTVDVPYKLAFTGSTAVLLVIALASFIVRSPEKYIYFAIGCTLFSIFFYFFFNEVRKRIRYVPDAAKKDFERAMYIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 339 - DNA, 933 bp, Wobblia lunata
  source 1..933: mol_type = other DNA
atgagcgcctgtcctacaccccctaccgtggtggcccccacagatcaaagccccaatcctaacggaggcaccacctcttgtgctcaggctgccccttctcctcagatgagcatgaattacaccctgcttgcaaccatcgacggcttcagcgccgtgatcctgcttggaagcggcgtggttttctggtactatagctggaggggaaccggcttcggagcccatcctcaccccaagaggaggcccctggagctgaacaccctgtctgctgccatcgctttcttaaccggcctgatgtacgtggccaaaacaagcgactttgcccgagacagcaccagcgagtttagcctgaacaggtactggtacagcgactacatcgttgtctgccccctgatcgtgatggacctgtgcttcaccgtggacgtgccttacaaactggcttttaccggcagcaccgctgttatgctggtgatcgccctggccagcttcattgtgagaagccccgagaaatacatttactttgccattggctgtaccctgtttagcattttcttttacttctttttcaatgaggtgagaaagagaatcaggtacgtgcccgacgccgccaagaaggacttggagagggccatgttcatcttcttcggcttctggcccatgttccccgttctgtggctgttaggcttccacaccaccggcgtgatcagcgaggaggcattcgccattattcacgccttcctggacatcacatgcaaaaccatcttcggcgtgttcatgctgaggtgtagactgcatatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 340 - protein, 311 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVVCPLIVMDLCFTVDVPYKLAFTGSTAVMLVIALASFIVRSPEKYIYFAIGCTLFSIFFYFFFNEVRKRIRYVPDAAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 341 - DNA, 921 bp, Wobblia lunata
  source 1..921: mol_type = other DNA
atgacaccccctaccgtggtggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacctggactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 342 - protein, 307 aa, Wobblia lunata
MTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 343 - DNA, 903 bp, Wobblia lunata
  source 1..903: mol_type = other DNA
atggcccccacagatcaaagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacctggactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 344 - protein, 301 aa, Wobblia lunata
MAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 345 - DNA, 888 bp, Wobblia lunata
  source 1..888: mol_type = other DNA
atgagccccaatcctaacggaggcaccacctcttgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacctggactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 346 - protein, 296 aa, Wobblia lunata
MSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 347 - DNA, 858 bp, Wobblia lunata
  source 1..858: mol_type = other DNA
atgtgtgctcaggctgccccttctcctcagatgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacctggactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 348 - protein, 286 aa, Wobblia lunata
MCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG

SEQ ID NO: 349 - DNA, 828 bp, Wobblia lunata
  source 1..828: mol_type = other DNA
atgagcatgaattacaccctgcttgcaaccatcgacggatttagcgccgtgattctgcttggaagcggcgtggtgttctggtactactcttggaggggaaccggctttggagcccacccccatcccaagagaagacccctggaactgaataccctgtctgccgccatcgccttcttaaccggcctgatgtacgtggccaagaccagcgactttgctagggacagcaccagcgagtttagcctgaacaggtactggtacctggactacatcgtgacatgccccctgatcgtgatggacctgtgcttcaccgtggacgtgccctacaagctggccttcaccggcagcaccgccgtgctgctggtgatcgccctcgccagcttcatcgtgaggagccccgagaagtacatctacttcgctatcggctgcaccctgtttagcattttcttctacttctttttcaacgaggtgagaaagaggatcaggtacgtgcccgactgcgccaagaaggacctggagagggccatgttcatcttcttcggcttctggcccatgttccccgtgctgtggctgctgggctttcacaccaccggcgtgatcagcgaggaggcctttgctatcattcacgccttcctagacattacctgcaagaccattttcggcgtgttcatgctgaggtgcagactgcacatcgaggactacgcctgggatgagctgaaaaggcagagacaggagatcgacgaggagaatcagaaaaccttgaccgccgctggcgccgatggcgccaccgtgatgatgcccgttgaagaagacgacccctacgctagcgga

SEQ ID NO: 350 - protein, 276 aa, Wobblia lunata
MSMNYTLLATIDGFSAVILLGSGVVFWYYSWRGTGFGAHPHPKRRPLELNTLSAAIAFLTGLMYVAKTSDFARDSTSEFSLNRYWYLDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFAIIHAFLDITCKTIFGVFMLRCRLHIEDYAWDELKRQRQEIDEENQKTLTAAGADGATVMMPVEEDDPYASG